SEQUENCE LISTING (18 sequences)

SEQ ID NO: 1 - DNA, 1906 bp, Bos taurus
caaggtcatgtcccaggaggagatagggaccgccctgcaccacaaacagctctgctccctcttataaagtagggcagcccagcccctggaagctcccaggatgcccggtccatctctctctctggccctggtgctgtcggccatgggggctctgctgaggccagggacccccagggaagaagtcttcagcacctcagccttgcccagggagcaggccacaggcagcggggcactcatctttcagcaagcctgggactggccactctccagtctctggctgccaggcagccctctggaccccctgtgcctggtgaccctgcatgggagtggcaacgggagcagggcccccctgcgggtggtgggggtcctgagcagctacgagcaggccttcctggaggctgtgcggcgcacccactggggcctgagtgacttgaccaccttcgcagtgtgccccgctggcaacgggcagcctgtgctgccccacctgcagcggctgcaggcatggctgggggagcccggggggcggtggctggtggtcctgcacctggaggaagtgacgtgggagccaacacccttgctgaggttccaggagcctccgcctggaggagccagccccccagagctggcgctgctggtggtgtacccagggcctggcctggaggtcactgtcaccggggctgggctacctggcacccagagcctctgcctgaccgcggactcggacttcctggccttggtcgtggaccacccggagggggcctggcgccggcctgggttagcccttaccctgcggcgccgtggaaatggtgcgctcctgagcactgcccagctgcaggcgctgctgttcggtgcggactcccgctgcttcacacgaaagaccccagccctgttactcttgctgccggcccggtcttcggcaccgatgcccgcgcacggtcggctggacttggtgcccttcccgcagcccagggcttccccggagccagaggaggcaccgcccagcgctgatcccttcctggagactctcacgcgcctggtgcgcgcgcttgcgggacccccggcccgagcctcgccaccgcggctggccttggacccgggcgcactggctggtttcccgcagggccaggtcaacctgtcggaccccgcggccctggagcgcctgctggacggcgaggagccgctgctgctgctgctgccgccgacggcagccaccaccggggtccccgcaacgccgcaaggtcccaagtcccctctgtgggccgcgggactagcgcgccgggtggctgccgagcttcaggcggtggccgccgagctgcgtgccctcccggggctgcctccagctgccccaccgctgctggcgcgcctgctggcactgtgcccgggaaacccagacagccccggcggcccgctgcgcgcgctgctgctgctcaaagcgctgcagggcctgcgcgctgagtggcgcgggcgggagcggagcggctctgcacgggcgcagcgcagcgccggggccgcggctgcagacgggccgtgcgctctgcgtgagctgagcgtagacctgcgggccgagcgctcggtgctcatccccgagacataccaggccaacaactgccagggggcctgcggctggcctcagtcggaccgcaacccgcgctacggcaaccacgtggtgctgctgctaaagatgcaggcccgcggcgccaccctggcgcgcccgccctgctgtgtgcccacagcctacaccggcaagctcctcatcagcctgtccgaggagcgcatcagtgcgcaccacgtcccaaacatggtggccaccgaatgcggctgccggtgacctcgcgccgtgctcctcgtgctgccccggcccgtatttattcggaccccgtcattgccccattaaacacgggaaggc

SEQ ID NO: 2 - protein, 575 aa, Bos taurus
MPGPSLSLALVLSAMGALLRPGTPREEVFSTSALPREQATGSGALIFQQAWDWPLSSLWLPGSPLDPLCLVTLHGSGNGSRAPLRVVGVLSSYEQAFLEAVRRTHWGLSDLTTFAVCPAGNGQPVLPHLQRLQAWLGEPGGRWLVVLHLEEVTWEPTPLLRFQEPPPGGASPPELALLVVYPGPGLEVTVTGAGLPGTQSLCLTADSDFLALVVDHPEGAWRRPGLALTLRRRGNGALLSTAQLQALLFGADSRCFTRKTPALLLLLPARSSAPMPAHGRLDLVPFPQPRASPEPEEAPPSADPFLETLTRLVRALAGPPARASPPRLALDPGALAGFPQGQVNLSDPAALERLLDGEEPLLLLLPPTAATTGVPATPQGPKSPLWAAGLARRVAAELQAVAAELRALPGLPPAAPPLLARLLALCPGNPDSPGGPLRALLLLKALQGLRAEWRGRERSGSARAQRSAGAAAADGPCALRELSVDLRAERSVLIPETYQANNCQGACGWPQSDRNPRYGNHVVLLLKMQARGATLARPPCCVPTAYTGKLLISLSEERISAHHVPNMVATECGCR

SEQ ID NO: 3 - DNA, 2834 bp, Homo sapiens
aaggtcgcggcagaggagataggggtctgtcctgcacaaacaccccaccttccactcggctcacttaaggcaggcagcccagcccctggcagcacccacgatgcgggacctgcctctcaccagcctggccctagtgctgtctgccctgggggctctgctggggactgaggccctcagagcagaggagccagctgtgggcaccagtggcctcatcttccgagaagacttggactggcctccaggcatcccacaagagcctctgtgcctggtggcactgggcggggacagcaatggcagcagctcccccctgcgggtggtgggggctctaagcgcctatgagcaggccttcctgggggccgtgcagagggcccgctggggcccccgagacctggccaccttcggggtctgcaacaccggtgacaggcaggctgccttgccctctctacggcggctgggggcctggctgcgggaccctggggggcagcgcctggtggtcctacacctggaggaaggtatgtggggcccagccccaagcttggcaccgccgtcttccttcaggtgggccgggtcctcctagggaagatcaggggctggcagagcccccaccctgggcagggaggctgtggtcttgttcctaggactgggttgcgggtccgtggcctggaaggtgggcaccacactctgtcctgtccccgaagcccagctcttagacttgcccctgcctcggtgccagggagagagctgctgccttctccccacccctgaagacgacgcagggctcggggccagtggaacccttcttcccacagccccagcctgttctcagggccgctggcctaagatactccctgcggggaaggggcttcatcgggcaccccaacccagagaccccagggcggcagccccacccacagcctcagacgcagcccctgcctgcccctgccgtcaccgctccctggctgcaggaaggcagctaagaggggcacccttgtcccccgcttgaggtcccctgcacagtggccagagcggcagggacagatcccaaagattcccggggggtgtggccttcaatggctcaggcgtcccctgctgtcccggctgcagtgacctgggagccaacaccctcgctgaggttccaggagcccccgcctggaggagctggccccccagagctggcgctgctggtgctgtaccctgggcctggccctgaggtcactgtgacgagggctgggctgccgggtgcccaggtaccagggagttgcatggggcagtgcccgggccgtggcggggggcatgaatttgttgcagggtctgcagtactgagaacagcgtagaaccagtggcgatgggaggaaggggaccggtagagcggggctgggtaagcctccatccagccgggctgagccctggtctccgcagagcctctgcccctcccgagacacccgctacctggtgttagcggtggaccgccctgcgggggcctggcgcggctccgggctggccttgaccctgcagccccgcggagagggtaggtccgcgtggagagggacggggagccgggtcgactgcccccgggcccccagcccctgagccagccgcgtgcccacccaccgcagactcccggctgagtaccgcccggctgcaggcactgctgttcggcgacgaccaccgctgcttcacacggatgaccccggccctgctcctgctgccgcggtccgagcccgcgccgctgcctgcgcacggccagctggacaccgtgcccttcccgccgcccaggtgcgcgcaggcaccgggacacggggcaggagcgggcgggggcggcgtggcctcgtggccgctctcaactcctccaattgcgggttccaggccatccgcggaactcgaggagtcgccacccagcgcagaccccttcctggagacgctcacgcgcctggtgcgggcgctgcgggtccccccggcccgggcctccgcgccgcgcctggccctggatccggacgcgctggccggcttcccgcagggcctagtcaacctgtcggaccccgcggcgctggagcgcctactcgacggcgaggagccgctgctgctgctgctgaggcccactgcggccaccaccggggatcctgcgcccctgcacgaccccacgtcggcgccgtgggccacggccctggcgcgccgcgtggctgctgaactgcaagcggcggctgccgagctgcgaagcctcccgggtctgcctccggccacagccccgctgctggcgcgcctgctcgcgctctgcccaggaggccccggcggcctcggcgatcccctgcgagcgctgctgctcctgaaggcgctgcagggcctgcgcgtggagtggcgcgggcgggatccgcgcgggccgggtcgggcacagcgcagcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggcccgtggggccgccctggcgcgcccaccctgctgcgtgcccaccgcctacgcgggcaagctgctcatcagcctgtcggaggaacgcatcagcgcgcaccacgtgcccaacatggtggccaccgagtgtggctgccggtgacccctgcgccgcgcggactcctgccccgagggtccggacgcgccccagctcgcgccccttcccatatttattcggaccccaagcatcgccccaataaagaccagcaagc

SEQ ID NO: 4 - protein, 560 aa, Homo sapiens
MRDLPLTSLALVLSALGALLGTEALRAEEPAVGTSGLIFREDLDWPPGIPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRAQRSAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 5 - DNA, 327 bp, Bos taurus
agcgccggggccgcggctgcagacgggccgtgcgctctgcgtgagctgagcgtagacctgcgggccgagcgctcggtgctcatccccgagacataccaggccaacaactgccagggggcctgcggctggcctcagtcggaccgcaacccgcgctacggcaaccacgtggtgctgctgctaaagatgcaggcccgcggcgccaccctggcgcgcccgccctgctgtgtgcccacagcctacaccggcaagctcctcatcagcctgtccgaggagcgcatcagtgcgcaccacgtcccaaacatggtggccaccgaatgcggctgccgg

SEQ ID NO: 6 - protein, 109 aa, Bos taurus
SAGAAAADGPCALRELSVDLRAERSVLIPETYQANNCQGACGWPQSDRNPRYGNHVVLLLKMQARGATLARPPCCVPTAYTGKLLISLSEERISAHHVPNMVATECGCR

SEQ ID NO: 7 - DNA, 327 bp, Homo sapiens
agcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggcccgtggggccgccctggcgcgcccaccctgctgcgtgcccaccgcctacgcgggcaagctgctcatcagcctgtcggaggaacgcatcagcgcgcaccacgtgcccaacatggtggccaccgagtgtggctgccgg

SEQ ID NO: 8 - protein, 109 aa, Homo sapiens
SAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 9 - DNA, 24 bp, Artificial Sequence
  Primer
cagagttcttgtctcctcagtgtt

SEQ ID NO: 10 - DNA, 24 bp, Artificial Sequence
  Primer
agagaatgacagcagtaggcatgt

SEQ ID NO: 11 - DNA, 18 bp, Artificial Sequence
  Primer
cttcattgacctcaacta

SEQ ID NO: 12 - DNA, 18 bp, Artificial Sequence
  Primer
ttcacacccatcacaaac

SEQ ID NO: 13 - DNA, 23 bp, Artificial Sequence
  Primer
cttcccacatagctcccttgtct

SEQ ID NO: 14 - DNA, 21 bp, Artificial Sequence
  Primer
gaacctccaggagtgccacag

SEQ ID NO: 15 - DNA, 23 bp, Artificial Sequence
  Primer
gttgatgccggtgaacgtgcaaa

SEQ ID NO: 16 - DNA, 23 bp, Artificial Sequence
  Primer
atcagctacaccagagacggaaa

SEQ ID NO: 17 - DNA, 22 bp, Artificial Sequence
  Primer
tcttaaactctgaagaagagac

SEQ ID NO: 18 - DNA, 20 bp, Artificial Sequence
  Primer
gtcttgcctgtatgtgatgg

(19) United States
(12) Patent Application Publication
(10) Pub. No.: US 2009/0304675 A1
(43) Pub. Date: Dec. 10, 2009

(54) METHOD OF MODULATION OF MULLERIAN INHIBITORY SUBSTANCE (MIS) RECEPTOR FOR THE TREATMENT OF NEURODEGENERATIVE DISEASES

(75) Inventors: Ian Stuart McLennan, Helensburgh (NZ); Kyoko Koishi, Helensburgh (NZ); Pei-Yu Wang, Warwick, RI (US)

Correspondence Address: HAYNES AND BOONE, LLP;IP Section, 2323 Victory Avenue, Suite 700, Dallas, TX, 75219, US

(73) Assignee: OTAGO INNOVATION LIMITED C/-TD SCOTT & CO., Denedin, NZ (NZ)

(21) Appl. No.: 12/091,231

(22) PCT Filed: Oct. 26, 2006
(86) PCT No.: PCT/NZ06/00275

§ 371 Date: Aug. 20, 2008

Related U.S. Application Data
(60) Provisional application No. 60/730,254, filed on Oct. 26, 2005.

(51) Int. Cl. A61K 39/395; A61K 38/16; A61K 31/7088; C12Q 1/02; C07K 16/00; C07H 21/04

(52) U.S. Cl. 424/130.1; 514/12; 514/44 R; 514/44 A; 514/13; 435/29; 530/387.1; 536/23.1; 536/24.5

(57) ABSTRACT

This invention relates to methods for modulating neuronal cell death or impairment, and methods for the treatment, prevention, and/or amelioration of symptoms of one or more conditions or diseases in a mammal caused by neuronal cell death or dysfunction.

### TECHNICAL FIELD

The present invention relates to a method of modulating neuronal cell survival or function and particularly although by no means exclusively to enhancing neuronal cell survival to treat neurodegenerative diseases.

### BACKGROUND OF THE INVENTION

Neurodegenerative disease encompasses a range of conditions that affect brain function, resulting from the deterioration of neurons.

Neurodegenerative diseases are generally progressive diseases associated with the progressive and persistent loss or dysfunction of neuronal cells and resulting in a decline in language, movement or recognition. These diseases can be divided into two groups: those related to movement and those related to memory and dementia.

Common neurodegenerative diseases include Alzheimer's disease, Parkinson's disease, Huntington's disease, Kennedy's disease and Multiple Sclerosis.

In the United States alone, five million people suffer from Alzheimer's disease, predicted to increase to 16 million by the year 2050. An additional 1.5 million people are thought to be afflicted with Parkinson's disease.

Neurodegenerative diseases appear to share many common molecular mechanisms with other brain disorders such as bipolar disorder and schizophrenia, which may hold the key to developing new treatments for these diseases. At present there are no cures and few treatments for neurodegenerative disease.

All references, including any patents or patent applications in this specification are hereby incorporated by reference as if explicitly set out in their entirety.

It is an object of the present invention to provide a method of modulating neuronal cell survival or function which goes at least some way towards addressing the problems disclosed above, or to at least provide the public with a useful choice.

### SUMMARY OF THE INVENTION

According to one aspect of the present invention there is provided a method of treating a condition or disease characterized by neuronal cell death or impairment in a patient in need thereof, said method comprising administering to said patient an effective amount of at least one Müllerian inhibitory substance (MIS) receptor agonist or antagonist.

In one embodiment said condition is characterized by neuronal cell death.

In one embodiment said condition is characterized by neuronal cell impairment.

According to another aspect there is provided a method of modulating neuronal cell function in a patient in need thereof, the method comprising the step of administering to said patient an effective amount of at least one Müllerian inhibitory substance receptor agonist or antagonist.

According to another aspect there is provided a method of enhancing neuronal cell survival in a patient in need thereof, the method comprising the step of administering to said patient an effective amount of at least one Müllerian inhibitory substance receptor agonist or antagonist.

In preferred embodiments the at least one Müllerian inhibitory substance receptor agonist or antagonist may induce neuronal cell differentiation and prevent the death and/or degeneration of neuronal cells both in vitro and in vivo.

The at least one Müllerian inhibitory substance receptor agonist or antagonist may also modulate neuronal cell function in neurons that are dysfunctional.

The present invention is preferably directed to the treatment, prevention or diagnosis of conditions where neurons are dysfunctional and/or degenerating, including neurodegenerative diseases such as Alzheimer's disease, Parkinson's disease, Huntington's disease, Friedreich's ataxia, cerebellar ataxia, other brain disorders such as bipolar disorder, epilepsy, schizophrenia, depression, mania, autism, ADHD, brain trauma injuries and stroke.

In one embodiment said neurons are located in regions of the brain comprising the cerebellum including but not limited to purkinje cells, the midbrain including but not limited to the substantia nigra, the forebrain including by not limited to the cerebral cortex, including but not limited to the caudate and putamen, the cerebrum, the hippocampus, the hypothalamus and the thalamus.

The present invention is also applicable to the treatment of patients who have menopause, suffer from an age- or disease related-dysfunction of the gonads or who have suffered unilateral or bilateral loss of a gonad.

In one embodiment the Müllerian inhibitory substance (MIS) receptor agonist is MIS; a functional derivative thereof; or an antibody or antibody fragment that binds to MIS, a functional derivative thereof, or a MIS receptor or receptor subunit.

In one embodiment the Müllerian inhibitory substance (MIS) receptor antagonist is an inactive variant of MIS; a MIS receptor; an antisense sequence; a siRNA; a ribozyme; or an antibody or antibody fragment that binds to MIS, a functional derivative thereof, or a MIS receptor or receptor subunit.

In one embodiment the Müllerian inhibitory substance receptor agonist or antagonist administered directly to a mammalian brain.

In one embodiment the Müllerian inhibitory substance receptor agonist or antagonist is administered systemically.

The Müllerian inhibitory substance receptor may be the type II receptor (MISRII), a type I receptor or a combination of both receptors. Preferably, the Müllerian inhibitory substance receptor is a type II receptor (MISRII).

Preferably the MISRII agonist or antagonist is Müllerian inhibitory substance (MIS), a functional derivative thereof, or an antibody or antibody fragment that specifically binds thereto.

In one embodiment the patient is a mammal, preferably a human.

In one embodiment the Müllerian inhibitory substance receptor agonist or antagonist comprises an at least 20 amino acid contiguous sequence from any one of SEQ ID NO:2, SEQ ID NO:4, SEQ ID NO:6, or SEQ ID NO:8.

In one embodiment the Müllerian inhibitory substance receptor agonist or antagonist comprises an at least 30 amino acid contiguous sequence from any one of SEQ ID NO:2, SEQ ID NO:4, SEQ ID NO:6, or SEQ ID NO:8.

In one embodiment the Müllerian inhibitory substance receptor agonist or antagonist comprises an at least 40 amino acid contiguous sequence from any one of SEQ ID NO:2, SEQ ID NO:4, SEQ ID NO:6, or SEQ ID NO:8.

In one embodiment the Müllerian inhibitory substance receptor agonist or antagonist comprises the amino acid sequence of any one of SEQ ID NO:2, SEQ ID NO:4, SEQ ID NO:6, or SEQ ID NO:8.

In one embodiment the Müllerian inhibitory substance receptor agonist or antagonist comprises the amino acid sequence of SEQ ID NO:4 or SEQ ID NO:8.

In one embodiment the Müllerian inhibitory substance receptor agonist or antagonist comprises a peptide or polypeptide sequence that is encoded by an at least 60 base pair contiguous nucleotide sequence selected from any one of SEQ ID NO:1, SEQ ID NO:3, SEQ ID NO:5, or SEQ ID NO:7.

In one embodiment the Müllerian inhibitory substance receptor agonist or antagonist comprises a peptide or polypeptide sequence that is encoded by an at least 90 base pair contiguous nucleotide sequence selected from any one of SEQ ID NO:1, SEQ ID NO:3, SEQ ID NO:5, or SEQ ID NO:7.

In one embodiment the Müllerian inhibitory substance receptor agonist or antagonist comprises a peptide or polypeptide sequence that is encoded by an at least 120 base pair contiguous nucleotide sequence selected from any one of SEQ ID NO:1, SEQ ID NO:3, SEQ ID NO:5, or SEQ ID NO:7.

In one embodiment the Müllerian inhibitory substance receptor agonist or antagonist comprises a peptide or polypeptide sequence that is encoded by an at least 150 base pair contiguous nucleotide sequence selected from any one of SEQ ID NO:1, SEQ ID NO:3, SEQ ID NO:5, or SEQ ID NO:7.

In one embodiment the Müllerian inhibitory substance receptor agonist or antagonist comprises a peptide or polypeptide sequence that is encoded by the nucleotide sequence of any one of SEQ ID NO:1, SEQ ID NO:3, SEQ ID NO:5, or SEQ ID NO:7.

In one embodiment the Müllerian inhibitory substance receptor agonist or antagonist is administered in conjunction with at least one additional active compound selected from the list comprising neurotrophic factors including but not limited to glial cell line-derived neurotrophic factor (GDNF), brain derived neurotrophic factor (BDNF), ciliary derived neurotrophic factor (CNTF), glutamate, and gonadal hormones including but not limited to estrogen, progesterone, androgen and synthetic equivalents thereof.

Another aspect of the present invention relates to the use of at least one Müllerian inhibitory substance receptor agonist or antagonist in the manufacture of a medicament for treating a condition or disease characterized by neuronal cell death or impairment in a patient in need thereof.

Another aspect of the present invention relates to the use of at least one Müllerian inhibitory substance receptor agonist or antagonist in the manufacture of a medicament for modulating neuronal cell function in a patient in need thereof.

Another aspect of the present invention relates to the use of at least one Müllerian inhibitory substance receptor agonist or antagonist in the manufacture of a medicament for enhancing neuronal cell survival in a patient in need thereof.

In one embodiment the Müllerian inhibitory substance receptor agonist or antagonist is formulated for delivery to a mammalian brain.

In one embodiment the Müllerian inhibitory substance receptor agonist or antagonist is formulated for systemic administration.

In one embodiment the medicament is formulated for simultaneous, separate or sequential administration with at least one additional active compound selected from the list comprising neurotrophic factors including but not limited to glial cell line-derived neurotrophic factor (GDNF), brain derived neurotrophic factor (BDNF), ciliary derived neurotrophic factor (CNTF), glutamate, and gonadal hormones including but not limited to estrogen, progesterone, androgen and synthetic equivalents thereof.

Another aspect of the present invention relates to a pharmaceutical composition comprising at least one Müllerian inhibitory substance receptor agonist or antagonist that modulates neuronal cell function in a patient in need thereof, together with a pharmaceutically acceptable carrier or excipient.

Another aspect of the present invention relates to a pharmaceutical composition comprising at least one Müllerian inhibitory substance receptor agonist or antagonist that enhances neuronal cell survival in a patient in need thereof, together with a pharmaceutically acceptable carrier or excipient.

In one embodiment the pharmaceutical composition is formulated for simultaneous, separate or sequential administration with at least one additional active compound selected from the list comprising neurotrophic factors including but not limited to glial cell line-derived neurotrophic factor (GDNF), brain derived neurotrophic factor (BDNF), ciliary derived neurotrophic factor (CNTF), glutamate, and gonadal hormones including but not limited to estrogen, progesterone, androgen and synthetic equivalents thereof.

Another aspect of the present invention relates to a method of diagnosing a condition or disease characterized by neuronal cell death or impairment or a predisposition to developing said condition or disease in a patient, the method comprising determining the level of MIS in a patient sample, wherein an alteration in the level of MIS compared to a control level indicates that the patient has a condition or disease characterized by neuronal cell death or impairment, or is at risk of developing said condition or disease.

Another aspect of the present invention relates to a method of diagnosing a condition or disease characterized by neuronal cell death or impairment or a predisposition to developing said condition or disease in a patient, the method comprising determining the expression level of MIS or a MIS receptor in a patient sample, wherein an alteration in the expression level of MIS or a MIS receptor compared to a control level indicates that the patient has a condition or disease characterized by neuronal cell death or impairment, or is at risk of developing said condition or disease.

In one embodiment the condition or disease is selected from the group comprising Alzheimer's disease, Parkinson's disease, Huntington's disease, Friedreich's ataxia, cerebellar ataxia, other brain disorders such as bipolar disorder, epilepsy, schizophrenia, depression, mania, autism and ADHD.

In one embodiment the condition or disease is the patient sample is selected from the list comprising tissue, blood, lymph, cerebrospinal fluid, urine, and ejaculate.

Another aspect of the present invention relates to a method of screening for a compound that modulates neuronal cell function or survival, the method comprising the steps of:
(a) contacting a test cell that expresses MIS or a MIS receptor with a test compound;
(b) determining the expression level of MIS or the MIS receptor; and
(c) selecting the compound that modulates the expression level compared to that in the absence of the test compound.

Another aspect of the present invention relates to a method of screening for a compound that modulates neuronal cell function or survival, the method comprising:
(a) contacting a test compound with MIS or a MIS receptor polypeptide;
(b) detecting the biological activity of the polypeptide; and either:
(c) selecting the compound that modulates the biological activity of the polypeptide in comparison with the biological activity detected in the absence of the compound; or
(d) selecting the compound that binds to the polypeptide.

In one embodiment the compound that alters expression or activity of MIS or a MIS receptor selected by the screening methods described above.

Another aspect of the present invention relates to a kit comprising a compound that alters expression or activity of MIS or a MIS receptor selected by the screening methods described above.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### BRIEF DESCRIPTION OF THE DRAWINGS

Further aspects of the present invention will become apparent from the following description which is given by way of example only and with reference to the accompanying drawings, wherein:

FIG. 1 and FIG. 2 show cultured motor neurons that have been stained using an antibody to MISRII. The neurons in FIG. 1 were from a control culture that lacked MIS. The cell on the left lacks a neurite and may be dead. The cell on the right has a single neurite, with no branching. The neuron in FIG. 2 was treated with MIS and has multiple highly branched neurites;

FIG. 3 illustrates the number of differentiated motor neurons (neurons with a neurite) in cultures of motor neurons that have been treated with various doses of MIS for 24 hours. Surviving motor neurons with axons were counted under a phase-contrast microscope;

FIG. 4 illustrates the total number of motor neurons in cultures without a growth factor (control, empty bar), 50 ng/ml of MIS or 50 ng/ml of GDNF. Total motor neuron counts of 50 ng/ml MIS or hGDNF treatment after 24 hours. Values are shown as mean±SEM (n=3);

FIG. 5 shows a section of a mouse cerebellum stained with cresyl violet to show neurons. The scale bar is 1 mm. “A” marks the locations of FIG. 6 and FIG. 7. “B” marks the location of FIG. 8 and FIG. 9;

FIG. 6 shows a higher magnification view of the cerebellum marked with an “A” in FIG. 5. The Purkinje cells are indicated by arrows. The scale bar is 30 μm;

FIG. 7 shows a section of cerebellum stained with an antibody to MISRII. The section is an adjacent section to that illustrated in FIG. 5. The illustrated region corresponds to that illustrated in FIG. 6. The arrows point to the Purkinje cells, which were the most intensely stained. Weak stain is present in the neurons of the molecular layer (arrowheads) and granule cell layer. The magnification is the same for FIG. 6;

FIG. 8 shows a higher magnification view of the region of cerebellum marked with a “B” in FIG. 5. The Purkinje cells are indicated by arrows. The magnification is the same as for FIG. 6. The section has been stained with cresyl violet to show neurons;

FIG. 9 shows a section of a cerebellum stained with an antibody to MISRII. The section is an adjacent section to that illustrated in FIG. 5. The illustrated region corresponds to that illustrated in FIG. 8. The arrows point to the Purkinje cells, which were the most intensely stained. Weak stain is present in the neurons of the granule cell layer. The axon tracts in the folium (F) and elsewhere have minimal staining. The magnification is the same as for FIG. 6;

FIG. 10 shows a section of cerebellum stained with non-immune IgG. The section is adjacent to those illustrated in FIG. 5, FIG. 6, FIG. 7, FIG. 8, and FIG. 9. No immunoreactivity is apparent, indicating that the stain illustrated in FIG. 7 and FIG. 9 is specific;

FIG. 11 shows a section of cerebellum stained with cresyl violet to stain neurons. The magnification is the same as for FIG. 5;

FIG. 12 shows a section of cerebellum from a RMSR mouse, stained with β-galactosidase to reveal the location of lacZ expression. The section is an adjacent section to that illustrated in FIG. 11. The RMSR mice produce lacZ under the control of the MISRII promoter. The lacZ stain therefore marks the location of the cells which produce MISRII. All of the neurons in the cerebellum were stained, with the intensity of the lacZ stain corresponding that observed with the anti-MISRII antibody (see FIG. 7 and FIG. 9). “A” marks the region of the cerebellum illustrated in FIG. 13. The section is the same magnification as FIG. 5 and FIG. 11;

FIG. 13 shows a section of cerebellum from a RMSR mouse, stained with β-galactosidase to reveal the location of lacZ expression. The section is a higher magnification view of the area marked with an “A” in FIG. 12. The Purkinje cells (arrows) were the most intensely stained, with lesser amounts of stain being present in the neurons of the granule cell and molecular layers. The axons in the folium (“F”) are stained. However, no stain was observed in the glia. The magnification is the same as for FIG. 6, FIG. 7, FIG. 8, FIG. 9, and FIG. 10;

FIG. 14 shows a section of the cerebral cortex of a murine brain stained with cresyl violet. The arrows point to the neurons, which are stained blue. The magnification is the same as for FIG. 6, FIG. 7, FIG. 8, FIG. 9, and FIG. 10;

FIG. 15 shows a section of cerebral cortex stained with an antibody to MISRII. The section is adjacent to that illustrated in FIG. 14. The arrows point to the neurons of the cortex, all of which were moderately stained by the anti-MISRII antibody. The magnification is the same as for FIG. 6, FIG. 6, FIG. 8, FIG. 9, and FIG. 10;

FIG. 16 shows a section of the hippocampus of a murine brain stained with cresyl violet. The arrows point to the neurons of the hippocampus. The magnification is the same as for FIG. 6, FIG. 7, FIG. 8, FIG. 9, and FIG. 10;

FIG. 17 shows a section of hippocampus stained with an antibody to MISRII. The section is adjacent to that illustrated in FIG. 16. The arrows point to the neurons of the hippocampus. The magnification is the same as for FIG. 6, FIG. 7, FIG. 8, FIG. 9, and FIG. 10; and

FIG. 18A and FIG. 18B show two photomicrographs of the choroid plexus in the fourth ventricle of a RMSR mouse. FIG. 18A has been stained with cresyl violet whereas FIG. 18B shows β-galactosidase stain (lacZ) to indicate the location of MISRII expression. The arrowheads point to the base of the cerebellum whereas the arrow points to choroid plexus within the ventricle. The sections illustrated in FIG. 18A and FIG. 18B are adjacent sections. The scale bar represents 500 μm.

FIG. 19 shows levels of GAPDH and MISRII mRNA in neurons from different parts of the brain in comparison to the testes.

FIG. 20A and FIG. 20B show the morphology of motor neurons in the lumbar lateral motor column of male wild type (A) and male MISRII^{-/-} (B) neonates. The scale bars=50 μm (A and B). FIG. 20C and FIG. 20D show the number (C) and nuclear size (D) of motor neurons in neonatal wild type and MISR^{-/-} mice of both sexes. “*” and “**” indicates that the mean value of the male and female data are significantly different, P=0.01 and P<0.001 respectively. The “#” and “##” indicates that the mean value of the wild-type and MISR^{-/-} data are significantly different, P=0.01 and P<0.001 respectively (Student's t-TEST, n=6 and 7 for each gender of wild-type and null mutant neonates, respectively).

FIG. 21 shows the number of motor neurons in the lumbar lateral motor column of male and female, wild type (+/+), MIS heterozygous (+/−), MIS knockout (−/−) mice. The results are mean±SEM of 7-9 mice. * The M^{+/+} was significantly different to both the female groups (p<0.0001). **M^{+/+} was significantly different to the MIS^{-/-} (p=0.002). There was no significant difference between the M−/− and the female groups (p>0.05).

FIG. 22 shows the nuclear volume of motor neurons in the lumbar lateral motor column of male (M), and female (F), wild type (+/+), MIS heterozygous (+/−), and MIS knockout (−/−) mice. The results are mean±SEM of 7-9 mice. None of the groups were statistically different to the M^{+/+} group (p>0.05)

### DETAILED DESCRIPTION

During development in males, MIS is exclusively produced by the testes. High levels of MIS are present in the blood of males from the eighth week of gestation until puberty, but the target of MIS has been unknown as MISRII has not previously been detected outside of the gonads and uterine precursor.

The inventors have previously identified that MIS is present in motor neurons (Wang P Y et al. (2005) Proc. Natl. Acad. Sci. USA; 102(45): 16421-5). In particular, the co-localization of MIS and MISRII in motor neurons was hypothesized as being consistent with MIS being an autocrine regulator of motor neuron function, or a paracrine regulator of motor neuron to motor neuron interactions. The MIS receptors are functional, the inventors subsequently showing that MIS is a motor neuron survival factor in vitro.

The inventors have now shown that the numbers and size of motor neurons are significantly higher in male nice than female mice. Moreover, this sexual dimorphism was absent in MIS^{-/-} and MISRII^{-/-} mice lacking MIS and MISRII, respectively.

The testes are the only known source of MIS in an embryo. Wang P Y et al. supra have shown that MIS is produced by adult neurons, but that embryonic neurons do not stain with anti-MIS antibodies. This indicates that embryonic neurons produce little or no MIS. Consequently, the observation that the number and size of motor neurons is altered in neonatal MIS^{-/-} and MISRII^{-/-} mice is strong evidence that MIS from the testes affects the brain. The known presence of MIS in male fetal and neonatal blood adds considerable weight to this conclusion.

In the adult, MIS ceases to be dimorphic (Lee et al., J. Clin. Endocrinol. Metab., 1996, 81(2):571-576; Wang et al., supra). Hence, its effects will cease to be male specific and should be important to both adult males and females.

The inventors have also surprisingly identified MIS receptors in a number of other neurons, including those of relevance to neurodegenerative diseases such as Alzheimer's disease, Parkinson's disease, Huntington's disease, Friedreich's ataxia, cerebellar ataxia, other brain disorders such as bipolar disorder, epilepsy, schizophrenia, depression, mania, autism, ADHD, brain trauma injuries and stroke. Consequently, agonists or antagonists of MIS receptors are of use in treating and diagnosing these conditions or diseases in which such neurons are dysfunctional and/or degenerating.

The inventors have found the highest expression of the MIS receptor MISRII in the choroid plexus, which is involved in the secretion and transport of molecules from the blood to the cerebrospinal fluid. This finding suggests that MIS is transported from the blood into the cerebrospinal fluid and further supports the role of MIS in the brain.

The present invention therefore provides methods of modulating neuronal cell survival or function in a patient by administering to said patient an effective amount of an agonist or antagonist of one or more MIS receptors.

Another aspect of the present invention relates to a method of treating a condition or disease characterized by neuronal cell death or impairment in a patient in need thereof, said method comprising administering to said patient an effective amount of at least one Müllerian inhibitory substance receptor agonist or antagonist.

In one embodiment the patient has menopause, suffers from an age- or disease related-dysfunction of the gonads or who has suffered unilateral or bilateral loss of a gonad.

The present method can also be used in vitro, for example, to induce cells in culture to differentiate to a neuronal phenotype. In one embodiment, the differentiated cells may be subsequently continued in culture, and can be used to provide useful in vitro assay systems for drug screening and development. In another embodiment, the differentiated cells may be used in vivo for transplantation.

### 1. DEFINITIONS

The term “antisense-oligonucleotides” as used herein encompasses both nucleotides that are entirely complementary to the target sequence and those having a mismatch of one or more nucleotides, so long as the antisense-oligonucleotides can specifically hybridize to the target sequence. For example, antisense-oligonucleotides of use in the present invention include polynucleotides that have an identity of at least about 70% to 75% or higher, at least about 80% to 85% or higher, at least about 90% to 95% or higher, at least about 96%, at least about 97%, at least about 98%, or at least about 99% or more sequence identity over a span of at least about 15, 20, 25, 30, 35, 40, 45, 50, 55, 60, 65, 70, 75, 80, 85, 90, 95, or 100 or more contiguous nucleotides to any of the nucleotide sequences set forth in SEQ ID NO:1, SEQ ID NO:3, SEQ ID NO:5, or SEQ ID NO:7.

The term “agonist or antagonist” means a biologically active agent that is capable of modulating a biological process. Agonists will usually be interpreted as inducing an augmentation of the biological process. Antagonists will usually be interpreted as inducing an inhibition of a biological process.

The term “analog” refers to a compound substantially similar in function to either the entire molecule or to a fragment thereof.

As used herein, a molecule is said to be a “chemical derivative” of another molecule when it contains additional chemical moieties not normally a part of the molecule, or when it does not contain chemical moieties that are normally a part of the molecule. Such moieties may impart a biological function with improved characteristics over the native compound (e.g., such a derivative may have a longer half-life than the native compound). The moieties may alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., Mack Publ., Easton, Pa., 1990.

The term “comprising” as used in this specification and the claims means “consisting at least in part of”. When interpreting statements in this specification and the claims which include that term, the features, prefaced by that term in each statement or claim, all need to be present but other features can also be present. Related terms such as “comprise” and “comprised” are to be interpreted in the same manner.

An “effective amount” is the amount required to confer therapeutic effect. The interrelationship of dosages for animals and humans (based on milligrams per meter squared of body surface) is described by Freireich, et al. (Cancer Chemother Rep. (1966) 50(4):219-44). Body surface area can be approximately determined from height and weight of the subject. See, e.g., Scientific Tables, Geigy Pharmaceuticals, Ardley, N.Y., 1970, 537. Effective doses also vary, as recognized by those skilled in the art, dependent on route of administration, excipient usage, and the like.

The term “enhancing neuronal cell survival” should be taken to mean treatments which induce or maintain neuron differentiation and prevent the degeneration and/or death of neurons compared to that observed in a patient in need thereof prior to treatment or in an untreated sample of an in-vitro culture.

A “fragment” of a polypeptide molecule such as MIS refers to a subsequence of the polypeptide that performs a function that is required for the biological activity and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

One preferred fragment is the transforming growth factor (TGF)-β-like C-terminal fragment of MIS of about 109 amino acids in length that is formed by the proteolytic (e.g., plasmin) cleavage of MIS and which migrates electrophoretically as a 12.5-kDa fragment (Pepinsky et al., J. Biol. Chem., 263:18961-4, 1988), hereby incorporated by reference. The Müllerian duct regression and antiproliferative bioactivities of MIS have previously been shown to reside in the C-terminal domain of MIS as described in U.S. Pat. No. 5,661,126, hereby incorporated by reference.

The term “carboxyl-terminal (C-terminal) fragment of MIS” is intended to include compounds and materials structurally similar to the about 12.5-kDa (about 25-kDa under non-reducing conditions) C-terminal fragment of MIS and of about 109 amino acids in length, resulting from proteolytic (e.g., plasmin) cleavage at residue 427 of the 535 amino acid human MIS monomer (residue 451 of SEQ ID NO:4) and the proteolytic (e.g., plasmin) cleavage site is at residue 443 of the 551 amino acid bovine MIS molecule (residue 466 of SEQ ID NO:2). In particular, “carboxyl-terminal (C-terminal) fragment of MIS” is intended to include the about 25-kDa homodimeric C-terminal fragment of MIS. The plasmin digested MIS has been shown to remain fully active in the organ culture assay (Pepinsky et al., J. Biol. Chem., 263:18961-4, 1988).

The nucleotide and amino acid sequences of the C-terminal fragment of bovine MIS are shown by SEQ ID NO:5 and SEQ ID NO:6, respectively. The nucleotide and amino acid sequences of the C-terminal fragment of human MIS are shown by SEQ ID NO:7 and SEQ ID NO:8, respectively.

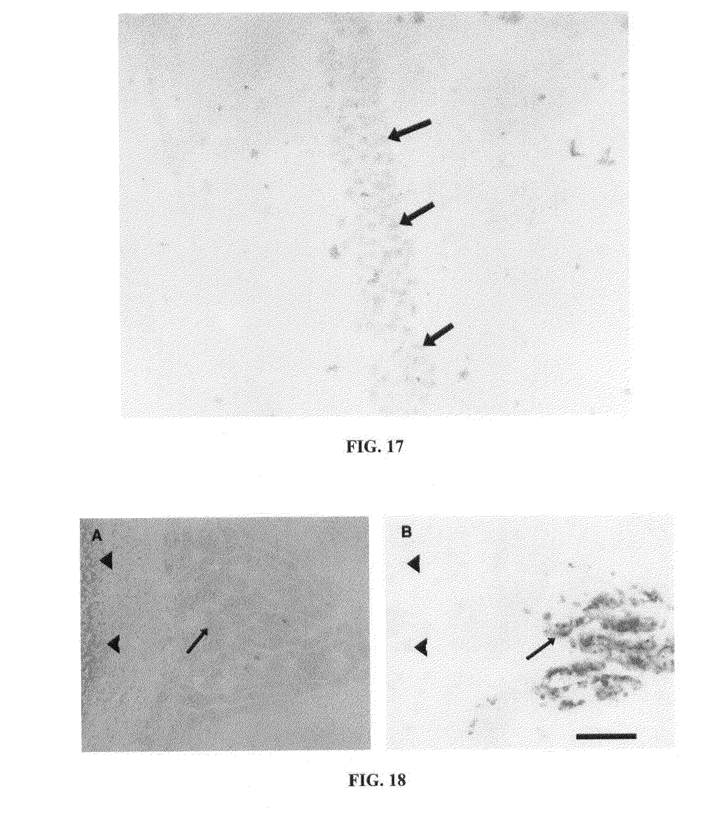

The C-terminal amino acid and nucleotide sequences for bovine MIS and human MIS are also shown in FIG. 17 and FIG. 18, respectively of U.S. Pat. No. 5,661,126.

A “fragment” of a polynucleotide sequence provided herein is a subsequence of contiguous nucleotides that is capable of specific hybridization to a target of interest, e.g., a sequence that is at least 15 nucleotides in length. The fragments preferably comprise at least about 15 nucleotides, preferably at least about 20 to 25 nucleotides, more preferably at least about 30 to 35 nucleotides, even more preferably at least about 40 to 50 nucleotides, still more preferably at least about 60 to 80 nucleotides and most preferably at least about 90 to 100 or more contiguous nucleotides that encode a MIS agonist and antagonist peptide or polypeptide for use in the invention. A fragment of a polynucleotide sequence can be used in antisense, gene silencing, triple helix or ribozyme technology, or as a primer, a probe, included in a microarray, or used in polynucleotide-based variant selection methods as discussed below.

A “functional derivative” of MIS is a compound which possesses biological activity (either functional or structural) that is substantially similar to the biological activity of MIS. The term “functional derivatives” is intended to include the “fragments,” “variants”, “analogs” or “chemical derivatives” of MIS.

The term “modulating neuronal cell function” should be taken to mean the artificial interference on neuronal cell function. This may include treatments that either increase or decrease total neuronal cell number; or that either increase or decrease neuronal cell receptor signal transduction to the cell, the sensitivity or total number of neuron receptors; when compared to the endogenous levels observed in a patient in need thereof prior to treatment.

The terms “Müllerian inhibitory substance” and “anti-Müllerian hormone” are synonymous and can be used interchangeably. The equivalent terms when used in relation to their respective receptors are to be similarly understood.

Müllerian inhibitory substance (MIS) is a member of the TGFβ superfamily of growth factors whose functions were believed to be restricted to reproductive tissues.

TGFβ superfamily members signal through a heteromeric receptor complex, containing a type I and type II receptor. Type II Müllerian inhibitory substance receptor (MISRII) has been identified and considered to be the only type II receptor for MIS. However, the identification of a type I receptor for MIS has been controversial, with a number of different type I receptors being suggested to mediate MIS signals. Suggested receptors include ALK-2, ALK-3 and ALK-6.

The nucleotide and amino acid sequences of bovine MIS (GenBank Accession Number M13151) are shown by SEQ ID NO:1 and SEQ ID NO:2, respectively. The nucleotide and amino acid sequences of human MIS (GenBank Accession Number K03474) are shown by SEQ ID NO:3 and SEQ ID NO:4, respectively. Both amino acid sequences set forth in SEQ ID NO:2 and SEQ ID NO:4 include a 24 amino acid leader peptide as amino acids 1 to 24.

The complete nucleotide sequences for bovine and human MIS are also disclosed in U.S. Pat. No. 5,047,336. A comparison of the amino acid sequence for human and bovine MIS is shown in Cate et al., Handbook of Experimental Pharmacology 95/11:184, edited by M. B. Sporn and A. B. Roberts, Springer-Verlag Berlin Heidelberg (1990), hereby incorporated by reference.

Endogenous MIS has been shown to be produced as a 140 kDa glycosylated disulfide-linked homodimer (Pepinsky et al., J. Biol. Chem., 263:18961-4, 1988). Under reducing conditions, the protein migrates on gel electrophoresis at an apparent molecular weight of 70 kDa. The protein can be proteolytically cleaved into two distinct fragments: a transforming growth factor (TGF)-β-like C-terminal fragment that migrates electrophoretically as a 12.5-kDa fragment; and a N-terminal fragment that migrates electrophoretically as a 57-kDa fragment (Pepinsky et al., J. Biol. Chem., 263:18961-4, 1988).

The term “patient” as used herein is preferably a mammal and includes humans, and non-human mammals such as cats, dogs, horses, cows, sheep, deer, mice, possum and primates and other domestic farm or zoo animals. Preferably, the mammal is a human.

The term “polynucleotide(s),” as used herein, means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polynucleotides possess biological activities that are the same or similar to those of the inventive polypeptides or polynucleotides. The term “variant” with reference to polynucleotides and polypeptides encompasses all forms of polynucleotides and polypeptides as defined herein.

Polynucleotide Variants

Variant polynucleotide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a specified polynucleotide sequence. Identity is preferably found over a comparison window of at least 20 nucleotide positions, at least 50 nucleotide positions, at least 100 nucleotide positions, or over the entire length of the specified polynucleotide sequence.

Polynucleotide sequence identity can be determined in the following manner. The subject polynucleotide sequence is compared to a candidate polynucleotide sequence using BLASTN (from the BLAST suite of programs, version 2.2.15 [October 2006]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters:

bl2seq -i nucleotideseq1 -j nucleotideseq2 -F F -p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants for use in the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.15 [October 2006]) from NCBI (ftp://ftp.ncbi.nih.gov/blast/).

The similarity of polynucleotide sequences may be examined using the following unix command line parameters:

bl2seq -i nucleotideseq1 -j nucleotideseq2 -F F -p tblastx

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰ less than 1×10⁻⁷⁰ less than 1×10⁻⁸⁰ less than 1×10⁻⁹⁰ less than 1×10⁻¹⁰⁰ less than 1×10⁻¹¹⁰ less than 1×10⁻¹²⁰ or less than 1×10⁻¹²³ when compared with any one of the specifically identified sequences.

Alternatively, variant polynucleotides for use in the present invention hybridize to a specified polynucleotide sequence, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C−log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides for use in the present invention also encompasses polynucleotides that differ from the sequences disclosed herein but that, as a consequence of the degeneracy of the genetic code, encode a polypeptide having similar activity to a polypeptide encoded by a specified polynucleotide. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.15 [October 2006]) from NCBI (ftp://ftp.ncbi.nih.gov/blast/) via the tblastx algorithm as previously described.

Polypeptide Variants

The term “variant” with reference to polypeptides encompasses naturally occurring, recombinantly and synthetically produced polypeptides. Variant polypeptide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a specified sequence. Identity is preferably found over a comparison window of at least 20 amino acid positions, at least 50 amino acid positions, at least 100 amino acid positions, or over the entire length of a polypeptide.

Polypeptide sequence identity can be determined in the following manner. The subject polypeptide sequence is compared to a candidate polypeptide sequence using BLASTP (from the BLAST suite of programs, version 2.2.15 [October 2006]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Polypeptide variants for use in the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.15 [October 2006]) from NCBI (ftp://ftp.ncbi.nih.gov/blast/). The similarity of polypeptide sequences may be examined using the following unix command line parameters:

bl2seq -i peptideseq1 -j peptideseq2 -F F -p blastp

Variant polypeptide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰ less than 1×10⁻⁶⁰ less than 1×10⁻⁷⁰ less than 1×10⁻⁸⁰ less than 1×10⁻⁹⁰ less than 1×10⁻¹⁰⁰ less than 1×10⁻¹¹⁰ less than 1×10⁻¹²⁰ or less than 1×10⁻¹²³ when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

A polypeptide variant for use in the present invention also encompasses that which is produced from the nucleic acid encoding a polypeptide, but differs from the wild type polypeptide in that it is processed differently such that it has an altered amino acid sequence. For example a variant may be produced by an alternative splicing pattern of the primary RNA transcript to that which produces a wild type polypeptide.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Notredame et al., J. Mol. Biol., 302:205-217, 2000) or PILEUP, which uses progressive, pairwise alignments. (Feng and Doolittle, J. Mol. Evol., 25:351, 1987).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, Nucl. Acids Res., 22:3583, 1994; Hofmann et al., Nucl. Acids Res., 27:215, 1999) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., Nucl. Acids Res., 30:235, 2002). Prosearch is a tool that enables a user to search a number of databases including SWISS-PROT, SWISS-2DPAGE, SWISS-3DIMAGE, and ENZYME, as well as other cross-referenced databases such as EMBL, GenBank, OMIM, Medline databases, etc., with a given sequence pattern or signature.

In addition to the computer/database methods described above, polypeptide variants may be identified by physical methods, for example by screening expression libraries using antibodies raised against MIS polypeptides used in the invention (Sambrook et al., Molecular Cloning: A Laboratory Manual, 2nd Ed. Cold Spring Harbor Press, 1987) or by identifying polypeptides from natural sources with the aid of such antibodies.

A receptor-ligand interaction may be determined using the yeast hybrid system, which involves the expression of chimeric proteins and their subsequent interaction within the yeast cell nucleus (Topcu and Dorden, Pharm. Res., 17:9, 2000). Unlike in-vitro biochemical techniques such as immunoprecipitation, the yeast hybrid system can detect in-vivo interactions, and does not require protein purification or antibody production. Accordingly, the yeast hybrid system is a powerful technique for identifying protein-protein interactions.

### 2. MIS RECEPTOR AGONISTS AND ANTAGONISTS

The present invention is directed to the surprising finding that MIS and MIS receptors are present in a number of neuronal cell types in the brain and that an MIS receptor agonist or antagonist is effective at modulating neuronal cell survival.

In preferred embodiments the MIS receptor agonist or antagonist is Müllerian inhibitory substance (MIS) or a C-terminal fragment thereof, functional derivatives or analogs thereof, or an antibody or antibody fragment that binds thereto.

In this context agonists include ligands that will bind to MIS, the C-terminal fragment of MIS, or a MIS receptor and enhance the signal transduction to the cell, the sensitivity or number of receptors. Examples of such ligands include MIS; a functional derivative thereof; a polypeptide; or an antibody or antibody fragment raised against MIS, the C-terminal fragment of MIS, or a MIS receptor or portions thereof.

In one embodiment MIS or functional derivatives thereof with the ability to bind to a MIS receptor may be provided as agonists to patients in order to enhance neuronal cell function and/or survival in accordance with the present invention.

In this context antagonists include ligands that will bind to MIS, the C-terminal fragment of MIS, or a MIS receptor and decrease the signal transduction to the cell, the sensitivity or number of receptors. Examples of such ligands include antibodies or fragments thereof capable of binding to MIS, the C-terminal fragment of MIS, or a MIS receptor or portions thereof; variants of MIS having the ability to bind to MIS receptor molecules but lacking other MIS activity; a MIS receptor molecule; and variants of a MIS receptor molecule which have the ability to bind to MIS.

In one embodiment antagonists of MIS, the C-terminal fragment of MIS, or a MIS receptor may be provided to patients in order to decrease neuronal cell function in accordance with the present invention, decreasing a patient's response to the presence of MIS.

Other agonists or antagonists include molecules that will affect the bioavailability of endogenous ligands. Such molecules include purified or recombinant MIS receptors or fragments thereof that will bind or scavenge endogenous ligands that naturally bind to MIS receptors in vivo, thereby modulating the activity of the MIS regulatory pathway.

The MISRII gene has been isolated from the rat (Baarends et al., Development, 120:189-197, 1994), rabbit (di Clemente et al., Mol. Endocrinol., 8:1006-1020, 1994), human (Imbeaud et al., Nat. Genet., 11:382-388, 1995) and mouse (Mishina et al., Genes Dev., 10:2577-2587, 1996). The complete coding sequence and mRNA of the mouse MISRII gene is available as GenBank Accession Numbers AF503863 and NM_{—}144547 respectively. The complete coding sequence and mRNA of the human MISRII gene are available as GenBank Accession Numbers AF172932 and NM_{—}020547, respectively.

In one embodiment the agonist or antagonist may be a recombinant protein comprising a MIS receptor binding or ecto-domain. Exon 2 of the MISRII gene seems to be essential for ligand binding, as shown in the rabbit receptor (di Clemente et al., Mol. Endocrinol., 8:1006-1020, 1994).

Recombinant MIS can be expressed in a protein expression system. The use of prokaryotic and eukaryotic expression systems are well known to those in the art (see generally Sambrook et al., Molecular Cloning. A Laboratory Manual, 2nd Ed. Cold Spring Harbor Press, 1987). For example, bacterial (e.g., E. coli), fungi (e.g., yeast), mammalian cells (e.g., CHO cells, COS cells) plant cells or insect cells (e.g., baculovirus transformed cells) expression systems can be used. U.S. Pat. No. 5,047,336 describes recombinant DNA techniques which can readily be used to produce MIS or a fragment thereof.

Methods for purifying non-recombinant MIS are also well known in the art and have been described in U.S. Pat. Nos. 4,404,188, 4,487,833 and 5,011,687

In one embodiment the MIS receptor agonist or antagonist comprises compounds that are structurally and/or functionally similar to MIS. Examples of such compounds include salts and functional derivatives of MIS as defined above.

In other embodiments the MIS receptor agonist or antagonist comprises an antibody or antibody fragment that is capable of binding an antigenic determinant of MIS, the C-terminal fragment of MIS, or a MIS receptor (i.e., that portion of a molecule (i.e., an epitope) that makes contact with a particular antibody or other binding molecule). Suitable antibodies and antibody fragments include, for example, intact antibodies (polyclonal, monoclonal, or chimeric), antibody fragments, antibody heavy chains, antibody light chains, single chain antibodies, single-domain antibodies (a VHH for example), Fab antibody fragments, Fc antibody fragments, Fv antibody fragments, F(ab′)₂ antibody fragments, Fab′ antibody fragments, and single-chain Fv (scFv) antibody fragments.

Antibody and antibody fragments for use in the present invention can be generated by a number of known artificial and natural processes as discussed below.

In one embodiment the antibody fragment may be Fab, Fab′, F(ab′)₂, Fv or single chain Fv (scFv), in which Fv fragments from H and L chains are ligated by an appropriate linker (Huston et al., Proc. Natl. Acad. Sci. USA, 85:5879-83, 1988). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co et al., J. Immunol., 152:2968-76, 1994; Better and Horwitz, Methods Enzymol., 178:476-96, 1989; Pluckthun and Skerra, Methods Enzymol., 178:497-515, 1989; Lanioyi, Methods Enzymol., 121:652-63, 1986; Rousseaux et al., Methods Enzymol., 121:663-9, 1986; Bird and Walker, Trends Biotechnol., 9:132-7, 1991).

An antibody may be modified by conjugation with a variety of molecules, such as polyethylene glycol (PEG). The modified antibody can be obtained by chemically modifying an antibody. These modification methods are conventional in the field.

Alternatively, an antibody may be obtained as a chimeric antibody, between a variable region derived from nonhuman antibody and the constant region derived from human antibody, or as a humanized antibody, comprising the complementarity determining region (CDR) derived from nonhuman antibody, the frame work region (FR) derived from human antibody, and the constant region. Such antibodies can be prepared using known art methods.

In brief, methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a MIS or MIS receptor polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL TDM adjuvant (moriophosphoryl Lipid A, synthetic trehalose dicorynomycolate). The immunization protocol may be selected by one skilled in the art without undue experimentation.

Intracellular antibodies are generally single chain antibodies herein they will comprise single chain antibodies which specifically bind a MIS protein or a MIS receptor. They may be used in gene therapy by incorporating the sequence encoding the antibody into a recombinant vector and administered to cells over-expressing a MIS protein or a MIS receptor to bind and inhibit its function. Methods for producing these antibodies are known in the art. (see for example Tanaka et al., Nucleic Acids Research 31 (5):e23 (2003)

Monoclonal antibodies may be prepared using hybridoma methods, such as those described by Kohler and Milstein (Nature, 256:495, 1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the MIS, the C-terminal fragment of MIS, or MIS receptor polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (see, e.g., Goding, Monoclonal Antibodies: Principles and Practice, Academic Press, pp. 59-103, 1986). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), that will prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [J. Immunol., 133:3001, 1984; Brodeur et al, Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the MIS, the C-terminal fragment of MIS, or MIS receptor polypeptide. Preferably, the binding specificity of monoclonal antibodies produced by the hybridoma cells is determined by immunoprecipitation or by an in vitro binding assay, such as radio linked immunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220, 1980.

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxyapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies for use in the invention can be readily isolated and sequenced using conventional procedures (e.g., by using oligonucleotide probes that are capable of binding specifically to genes encoding the heavy and light chains of murine antibodies. The hybridoma cells serve as a preferred source of such DNA. Once isolated, the DNA may be placed into expression vectors, which are then transfected into host cells such as simian COS cell Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody for use in the invention, or can be substituted for the variable domains of one antigen-combining site of an antibody to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

The antibodies for use in the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from nonhuman immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues.

Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329, 1988; and Presta, Curr. Op. Struct. Biol., A:593-596, 1992].

Methods for humanizing non-human antibodies are well known in the art. Generally, a humanized antibody has one or more amino acid residues introduced into it from a source which is non human. These non-human amino acid residues are often referred to as “import’ residues, which are typically taken from an “import” variable domain. Humanization can be essentially performed following the method of Winter and co-workers [Jones et al., Nature, 321:522-525, 1986; Riechmann et al., Nature, 332:323-327, 1988; Verhoeyen et al, Science, 239:1534-1536, 1988], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol Biol., 222:581 (1991)]. The techniques of Cole et al. and Boerner et al. are also available for the preparation of human monoclonal antibodies (Cole et al., Monoclonal Antibodies and Cancer Therapy Alan R. Liss, p. 77 (1985) and Boerner et al., J. Immunol., 147(1):86-95 (1991)]. Similarly, human antibodies can be made by introducing of human immuoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology, 10:779-783 (1992); Lonberg et al., Nature, 368 856-859 (1994); Morrison, Nature, 368:812-13 (1994); Fishwild et al., Nature Biotechnology, 14:845-51 (1996); Neuberger, Nature Biotechnology, 14:826 (1996); Lonberg and Huszar, Intern. Rev. Immunol., 13:65-93 (1995).

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

Bi-specific antibodies are monoclonal, preferably human or humanized, antibodies that have binding specificities for at least two different antigens. In the present case, one of the binding specificities is for MISRII, the other one is for any other antigen, and preferably for a cell-surface protein or receptor or receptor subunit.

Methods for making bi-specific antibodies are known in the art. Traditionally, the recombinant production of bi-specific antibodies is based on the co-expression of two immunoglobulin heavy chain light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bi-specific structure. The purification of the correct molecule is usually accomplished by affinity chromatography steps. Similar procedures are disclosed in PCT Intl. Pat. Appl. Publ. No. WO 93/08829, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bi-specific antibodies see, for example, Suresh et al., Methods Enzymol., 121:210 (1986).

According to another approach described in PCT Intl. Pat. Appl. Publ. No. WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bi-specific antibodies can be prepared as full length antibodies or antibody fragments (e.g., F(ab′)₂ bi-specific antibodies). Techniques for generating bi-specific antibodies from antibody fragments have been described in the literature. For example, bi-specific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bi-specific antibody. The bi-specific antibodies produced can be used as agents for the selective immobilization of enzymes.

Fab′ fragments may be directly recovered from E. coli and chemically coupled to form bi-specific antibodies. Shalaby et al., (J. Exp. Med., 175:217-225, 1992) describe the production of a fully humanized bi-specific antibody F(ab′) molecule. Each Fab′ fragment was separately secreted from E. coli and subjected to directed chemical coupling in vitro to form the bi-specific antibody. The bi-specific antibody thus formed was able to bind to cells overexpressing the ErbB2 receptor and normal human T cells, as well as trigger the lytic activity of human cytotoxic lymphocytes against human breast tumor targets.

Various techniques for making and isolating bi-specific antibody fragments directly from recombinant cell culture have also been described. For example, bi-specific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., (Proc. Natl. Acad. Sci. USA, 90:6444-6448, 1993) has provided an alternative mechanism for making bi-specific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bi-specific antibody fragments by the use of single-chain Fv (sFv) dimers has also been reported (See, e.g., Gruber et al., J. Immunol., 152:5368, 1994).

Antibodies with more than two valencies are contemplated. For example, trispecific antibodies can be prepared (see, e.g., Tutt et al., J. Immunol., 147:60, 1991).

Exemplary bi-specific antibodies may bind to two different epitopes on a given MIS receptor polypeptide herein. Alternatively, an anti-MIS receptor polypeptide arm may be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRII (CD 16) so as to focus cellular defense mechanisms to the cell expressing the particular MIS receptor polypeptide. Bi-specific antibodies may also be used to localize cytotoxic agents to cells which express a particular MIS receptor polypeptide. These antibodies possess a MISR-binding arm and an arm which binds a cytotoxic agent or a radionuclide chelator, such as EOTUBE, DPTA, DOTA, or TETA. Another bi-specific antibody of interest binds the MIS receptor polypeptide and further binds tissue factor (TF).

Antiidiotypic antibodies can also be used in the therapies discussed herein, to induce an immune response to cells expressing a MIS protein or a MIS receptor. Production of these antibodies is also well known (see for example Wagner et al., Hybridoma 16:33-40 (1997)).

Heteroconjugate antibodies are also within the scope of the present invention. Heteroconjugate antibodies are composed of two covalently joined antibodies. Such antibodies have, for example, been proposed to target immune system cells to unwanted cells (see, e.g., U.S. Pat. No. 4,676,980), and for treatment of HIV infection (PCT Intl. Pat. Appl. Publ. No. WO 91/00360; and PCT Intl. Pat. Appl. Publ. No. WO 92/200373). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include immunothiolate and methyl-4-mercaptobutyrimidate and those disclosed, for example, in U.S. Pat. No. 4,676,980.

It may be desirable to modify an antibody with respect to effector function, so as to enhance, e.g., the effectiveness of the antibody. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC) (See, e.g., Caron et al., J. Exp Med., 176:1191-1195, 1992 and Shopes, J. Immunol., 148:2918-2922, 1992). Homodimeric antibodies may also be prepared using heterobifunctional cross-linkers as described in Wolff et cii. Cancer Research, 53:2560-2565, 1993. Alternatively, an antibody can be engineered that has dual Fc regions and may thereby have enhanced complement lysis and ADCC capabilities. See, e.g., Stevenson et al., Anti-Cancer Drug Design, 3:219-230, 1989.

The at least one Müllerian inhibitory substance receptor agonist or antagonist may also alter the expression level or activity of MIS, the C-terminal fragment of MIS, or a MIS receptor in the patient. This may be by promoting expression, or administration of composition comprising a polynucleotide coding for a Müllerian inhibitory substance receptor agonist or antagonist. Alternatively, this may be by inhibiting expression. Whether promotion or inhibition of expression levels is appropriate will depend on the desired effect. Without wishing to be bound by theory, both over- and under-expression of polynucleotides are believed to be possible at this time.

Algorithms known in the art as discussed above can be used to determine the identity. Furthermore, derivatives or modified products of the antisense-oligonucleotides can also be used as antisense-oligonucleotides in the present invention. Examples of such modified products include lower alkyl phosphonate modifications such as methyl-phosphonate-type or ethyl-phosphonate-type, phosphorothioate modifications and phosphoroamidate modifications.

Antisense-oligonucleotides corresponding to the nucleotide sequence of MIS, the C-terminal fragment of MIS, or a MIS receptor can be used to reduce expression in situations where that is required. These anti-sense-oligonucleotides may act by binding to the polypeptides coding for MIS, the C-terminal fragment of MIS, or a MIS receptor or mRNAs corresponding thereto and thereby inhibiting the transcription or translation thereof, promoting the degradation of the mRNAs, and/or inhibiting the expression of the proteins encoded by the nucleotides, and finally inhibiting the function of the proteins.

In one embodiment expression may be inhibited by administering an antisense composition to the patient, the composition comprising a polynucleotide sequence complementary to SEQ ID NO:1, SEQ ID NO:3, SEQ ID NO:5, or SEQ ID NO:7.

The nucleic acids that inhibit one or more gene products also include small interfering RNAs (siRNA) comprising a combination of a sense strand nucleic acid and an antisense strand nucleic acid of a nucleotide sequence coding for MIS, the C-terminal fragment of MIS, or a MIS receptor. The term “siRNA” refers to a double stranded RNA molecule which prevents translation of a target mRNA. Standard techniques of introducing siRNA into the cell can be used in the treatment or prevention of the present invention, including those in which DNA is a template from which RNA is transcribed. The siRNA is constructed such that a single transcript has both the sense and complementary antisense sequences from the target gene, e.g., a hairpin.

The nucleotide sequence of siRNAs may be designed using a siRNA design computer program available from the Ambion website

(http:/Iwww.ambion.com/techlib/misc/siRNA_finder.html). Nucleotide sequences for the siRNA are selected by the computer program based on the following protocol:

Selection of siRNA Target Sites:

1. Beginning with the AUG start codon of transcript, scan downstream for AA dinucleotide sequences. Record the occurrence of each AA and the 3′ adjacent 19 nucleotides as potential siRNA target sites. Tusehi, et al. recommend not to design siRNA against the 5′ and 3′ untranslated regions (UTRs) and regions near the start codon (within 75 bases) as these may be richer in regulatory protein binding sites, and thus the complex of endonuclease and siRNAs that were designed against these regions may interfere with the binding of UTR-binding proteins and/or translation initiation complexes.

2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/

3. Select qualifying target sequences for synthesis. On the website of Ambion, several preferable target sequences can be selected along the length of the gene for evaluation.

The siRNAs inhibit the expression of MIS, the C-terminal fragment of MIS, or a MIS receptor protein and is thereby useful for suppressing the biological activity of proteins. In one embodiment expression is inhibited by administering a siRNA composition to the patient the composition reducing the expression of a sequence set forth in SEQ ID NO:1 or SEQ ID NO:3.

It is contemplated that the agonists or antagonists identified will be tested for biological activity in an animal model or in an in vitro model and suitably active compounds formulated into pharmaceutical compositions.

A suitable activity assay is the rat Müllerian duct regression organ culture assay of Donahoe (Donahoe et al., J. Surg. Res. 23:141-148 (1977) that measures the ability of MIS to promote regression of the Müllerian duct.

Other screening assays may also be used. In one embodiment the screening assay comprises the steps of:
(a) contacting a test cell that expresses MIS or a MIS receptor with a test compound;
(b) determining the expression level of MIS or the MIS receptor; and
(c) selecting the compound that modulates the expression level compared to that in the absence of the test compound.

In another embodiment the screening assay comprises the steps of:
(a) contacting a test compound with MIS or a MIS receptor polypeptide;
(b) detecting the biological activity of the polypeptide; and either:
(c) selecting the compound that modulates the biological activity of the polypeptide in comparison with the biological activity detected in the absence of the compound; or
(d) selecting the compound that binds to the polypeptide.

### 3. MIS RECEPTOR AGONIST OR ANTAGONIST APPLICATIONS

The present invention is directed to the treatment or prevention of conditions where neurons are dysfunctional and/or degenerating, including neurodegenerative diseases such as Alzheimer's disease, Parkinson's disease, Huntington's disease, Friedreich's ataxia, cerebellar ataxia, other brain disorders such as bipolar disorder, epilepsy, schizophrenia, depression, mania, autism, ADHD, brain trauma injuries and stroke.

In one embodiment said neurons are located in regions of the brain comprising the cerebellum including but not limited to purkinje cells, the midbrain including but not limited to the substantia nigra, the forebrain including by not limited to the cerebral cortex, including but not limited to the caudate and putamen, the cerebrum, the hippocampus, the hypothalamus and the thalamus.

Therapeutic formulations containing a compound, antisense sequence, siRNA, ribozyme, polypeptide or antibody for use in the invention may be prepared by mixing the active molecule with optional pharmaceutically acceptable carriers, excipients or stabilizers (Remington's Pharmaceutical Sciences 16th edition, Osol, A. Ed. [1980]), The composition may be formulated for oral administration (eg capsules, tablets, lozenges, powders, syrups, and the like), for parenteral administration (eg intravenous solutions, subcutaneous, intramuscular or suppository formulations), for topical administration (eg creams, gels), for inhalation (eg intranasal, intrapulmonary).

Therapeutic formulations containing MIS receptor agonists or antagonists can be prepared for storage by mixing the active molecule having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions.

Acceptable carriers, excipients, or stabilizers are well known in the art. They must be nontoxic to recipients at the dosages and concentrations employed, and include buffers such as phosphate, citrate, and other organic acids, water, oils, particularly olive, sesame, coconut and mineral and vegetable oils, antioxidants such as ascorbic acid and methionine; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including lactose, glucose, mannose, or dextrins; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; chelating agents such as EDTA; sugars such as sucrose, mannitol, trehalose or sorbitol; salt-forming counter-ions such as sodium; metal complexes (e.g., Zn-protein complexes); non-ionic surfactants such as TWEEN™, PLURONICS™ or polyethylene glycol (PEG).

For tablets, diluents such as carbonates (eg sodium and calcium) phosphates (such as calcium phosphate) or lactose are commonly used with preservatives, antioxidants, granulating and disintegrating agents (eg corn starch), binding agents such as starch, and lubricating agents such as stearic acid and magnesium stearate. Tablets may be coated to facilitate ingestion, stability or disintegration.

Injectable formulations are usually prepared with wetting and suspending agents as well as diluents or vehicles such as saline.

Any conventional technologies may be employed to produce tablets, topical and intravenous formulations, syrups, oil-in-water emulsifiers, inhalants and the like (Remington's supra

Compounds identified by the screening assays disclosed herein can be formulated in an analogous manner, using standard techniques well known in the art.

The formulations to be used for in vivo administration must be sterile. This is readily accomplished by filtration through sterile filtration membranes.

Lipofections or liposomes can also be used to deliver the compounds into cells. Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g., Marasco et al., Proc. Nat. Acad. Sci. USA, 90:7889-7893, 1993).

The formulation herein may also contain more than one active compound as necessary for the particular indication being treated, preferably those with complementary activities that do not adversely affect each other. Such molecules are suitably present in combination in amounts that are effective for the purpose intended.

For example, when used in the treatment of patients who have menopause, who have suffered unilateral or bilateral loss of a gonad and/or whose gonads have age- or disease-related dysfunction, a MIS receptor agonist or antagonist may be used in combination with gonadal hormones such as estrogen, progesterone, androgens or their known art synthetic equivalents in hormone replacement therapies.

In other embodiments the Müllerian inhibitory substance receptor agonist or antagonist is administered in conjunction with at least one additional neurotrophic factor selected from the list comprising neurotrophic factors including but not limited to glial cell line-derived neurotrophic factor (GDNF), brain derived neurotrophic factor (BDNF), ciliary derived neurotrophic factor (CNTF) and glutamate.

In further embodiments the additional active compound may be used to remove or inactivate molecules that would otherwise bind/inactivate the Müllerian inhibitory substance receptor agonist or antagonist.

The active compounds may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ‘y-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

A number of studies describe the use of MIS in the treatment of varying disease states and such delivery methods can be adapted for use in the present invention.

U.S. Pat. No. 5,661,126 describes the use of MIS for treating certain tumours using an effective amount of MIS. Compositions comprising MIS, the C-terminal fragment of MIS or their functional derivatives are described, as are gene therapy treatments for inhibiting the growth of such cancers.

U.S. Pat. No. 6,692,738 describes delivery systems comprising implantable matrices seeded with cells genetically engineered to express MIS. Such implants were shown to produce bioactive MIS in supraphysiologic concentrations, reducing ovarian tumor growth in mice. Similar in-vivo delivery systems are applicable to the present invention.

A number of gene therapy methods relating to the administration of nucleic acid sequences encoding MIS have also been described in U.S. Pat. No. 6,673,352.

For example, cells from a patient may be engineered ex vivo with a polynucleotide (DNA or RNA) comprising a promoter operably linked to an MIS polynucleotide as set forth in SEQ ID NO:1 or SEQ ID NO:3 or C-terminal fragments thereof, with the engineered cells then being provided to a patient to be treated with the polypeptide. Alternatively, the gene constructs bearing the MIS polynucleotide may be directly administered to the cells of an animal by any method that delivers injectable materials.

In preferred embodiments the modes of administration may involve the use of gene constructs operatively linked to a neuron- or glia-specific promoter such as neuron-specific enolase (neuron-specific) or glial fibrillary acidic protein (GFAP) (Harrop J S et al., Spine (2004). 29(24):2787-92; Navarro V et al., Gene Therapy, (1999) 6(11):1884-92)

The precise nature of the carrier or other material will be dependent upon the desired nature of the pharmaceutical composition, and the route of administration e.g., oral, intravenous, cutaneous, subcutaneous, intradermal, intramuscular, intra-articular, intrasynovial, intraperitoneal or intracerobrospinal.

In immunoadjuvant therapy, the pharmaceutical composition will be in the form of a parenterally acceptable aqueous solution which is pyrogen-free and has suitable pH, isotonicity and stability.

When treating neurodegenerative diseases or other brain disorders, brain specific modes of administration may be used such as direct injection into the brain, including intrathecal or intraventricular injection. Alternatively, gene constructs may obtain entry to the brain by retrograde axonal transport after injection into the periphery, for example following intramuscular or intravenous injection.

MIS, the C-terminal fragment thereof or their functional derivatives can also be administered systemically and modulate neuronal cell function and survival via the capillaries of the blood-brain barrier or via the choroid plexus.

Despite the classical perception that protein factors cannot pass the blood-brain barrier, other TGFβ molecules which share a number or characteristics of molecular structures with MIS have been known to cross through the blood-brain barrier (Chang et al, Stroke, 34:558-564, 2003, and McLennan et al., Neuropharmacology, 48:274-282, 2005).

Drug delivery across the blood-barrier has been a focal point of pharmaceutical science that is directly linked to the development of new therapeutic treatment. A number of methods have been tested at clinical trial levels and reviewed by Kemper et al., Cancer Treatment Rev., 30:415-423, 2004.

The finding by the inventors of high levels of expression of the MIS receptor MISRII in the choroid plexus offers an alternative route for MIS to be transported from the blood into the cerebrospinal fluid.

Administration of a pharmaceutical composition in accordance with the invention is preferably in a “therapeutically effective amount”, this being sufficient to show the desired benefit to the individual. The actual amount administered, and rate and time-course of administration, will depend on the nature and severity of the underlying condition. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Oslo, A. (Ed), 1980.

The effective amount may vary depending upon criteria such as the age, weight, physical condition, past medical history, and sensitivity of the recipient. The effective amount will also vary depending on the route of administration, for example oral, intravenous, intramuscular, subcutaneous, local, or by direct application to the brain.

In methods of enhancing neuronal cell survival in vitro, the method comprises culturing the neuronal cells in the presence of at least one MIS receptor agonist or antagonist. Neuronal cell cultures can be prepared following protocols well known in the art and as described herein in the Examples. Immediately following seeding, MIS can be added to promote neuron growth and differentiation using standard cell culture techniques.

In some embodiments, it is preferable to achieve a serum or medium concentration of at least 1 ng/ml of MIS or a C-terminal fragment thereof. Preferably the concentration ranges from about 1 ng/ml to about 20 μg/ml of MIS or a C-terminal fragment thereof, preferably from about 10 ng/ml to about 500 ng/ml, preferably from about 50 ng/ml to about 100 ng/ml. It is expected that the agonists or antagonists may be therapeutic across a range of doses, with a preference towards dose regimes that reproduce the levels of MIS in male fetuses and boys.

The concentration of MIS in the serum of boys before puberty is 40-90 ng/ml. It drops to a low level 3.7˜6.9 ng/ml in adults (Lee et al., J. Clin. Endocrinol. Metab., 1996, 81(2):571-576).

Therapeutically effective doses of MIS have been described in the literature for the treatment of varying disease states and may be used as a starting point when determining the appropriate amount of agonist or antagonist for use in the invention.

For the direct application to a tumor, U.S. Pat. No. 6,673,352 describes a serum concentration of MIS protein or the C-terminal fragment thereof ranging from 1 ng/ml to about 20 μg/ml. In the treatment of excess androgen states, a single injection of 1 mg MIS was found to lower testosterone concentration in rats.

U.S. Pat. No. 5,198,420 describes a subcutaneous injection of 1 μg MIS for the treatment of neonatal respiratory distress syndrome, equating to a serum concentration of approximately 1 μg/ml.

U.S. Pat. No. 6,692,738 describes implants of varying sizes, each comprising a matrix seeded with cells genetically engineered to express MIS and implanted into the ovarian pedicle of mice. 14 days after implantation, the serum levels of MIS measured 100-500 ng/ml and at 28 days the levels measured 7-10 μg/ml. Implants secreting MIS were shown to significantly reduce ovarian tumor growth in mice compared with the implant alone.

Gupta et al. (2005) describes the use of MIS to suppress tumor growth in the C3T(1)T antigen transgenic mouse mammary carcinoma model. In these studies, 20 μg MIS was administered per animal for five days a week with a treatment-free interval of 2 days over a six week period. MIS was shown to inhibit the growth of spontaneously arising mammary tumors.

In another aspect there is provided a method of diagnosing a condition or disease characterized by neuronal cell death or impairment or a predisposition to developing said condition or disease in a patient.

Such methods comprise determining the level of MIS in a patient sample, wherein an alteration in the level of MIS compared to a control level indicates that the patient has said condition or disease, or is at risk of developing said condition or disease.

Alternatively, such methods may comprise determining the expression level of MIS or a MIS receptor in a patient sample, wherein an alteration in the level of MIS compared to a control level indicates that the patient has said condition or disease, or is at risk of developing said condition or disease.

A “control level” refers to the level of MIS or MIS receptor protein or the level of MIS or MIS receptor expression detected in a normal healthy individual, or a level determined based on a population of individuals not known to be suffering from said condition or disease. The control level may be a single protein level or expression pattern derived from a single reference population or may be a plurality of levels or expression patterns. For example, the control level can be a database of levels or expression patterns from previously tested samples. Another example may be a ratiometric measure between a reference marker and the level or expression pattern.

A sample used herein comprises a biological sample derived from a patient to be screened. The biological sample may be any suitable sample known in the art in which the presence of MIS or a MIS receptor protein, or the expression of MIS or a MIS receptor can be detected.

Included are individual cells and cell populations obtained from bodily tissues or fluids. Examples of suitable body fluids to be tested are blood, lymph, cerebrospinal fluid, urine and ejaculate. Samples from healthy individuals may also be tested. A normal healthy individual is one with no clinical symptoms and preferably under 30 years of age.

The level of protein or expression level of MIS or a MIS receptor can be considered to be altered if the level differs from the control level by more than 5%, more than 10%, more than 20% more than 30%, more than 40% preferably by more than 50% or more compared to the control level.

For example, the condition or disease may arise in males through failure of MIS expression to drop from high pre-puberty levels to the low post-puberty levels.

The presence of MIS or a MIS receptor or their level of expression in the sample may be determined according to methods known in the art such as ELISA, Southern Blotting, Northern Blotting, FISH or quantative PCR to quantitate the transcription of mRNA [(Thomas, Pro. NAH, Acad. Sci. USA 77: 5201-5205 1980), (Jain K K, Med Device Technol. 2004 May; 15(4):14-7)], dot blotting, (DNA analysis) or in situ hybridization using an appropriately labelled probe, based on the marker sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labelled and the assay may be carried out where the duplex is bound to a surface, so that when the duplex is formed on the surface the presence of the antibody bound to the duplex can be detected.

MIS or a MIS receptor levels or expression patterns may alternatively be measured by immunological methods such as immunohistochemical staining of cells or tissue sections and assay of cell culture or body fluids to quantitate directly the level. Antibodies useful for immunohistochemical staining and/or for assay of sample fluids are preferably either monoclonal or polyclonal and methods for their generation have been discussed above.

The present methods can also be used in vitro, for example, to induce cells in culture to differentiate to a neuronal phenotype. In one embodiment, the differentiated cells may be subsequently continued in culture, and can be used to provide useful in vitro assay systems for drug screening and development as described above. In another embodiment, the differentiated cells may be used in vivo for transplantation.

The invention also provides a kit containing materials e.g., comprising one or more compounds useful for the diagnosis or treatment of diseases or conditions characterized by neuronal cell death or impairment.

In other embodiments the kit comprises a MIS agonist or antagonist that alters expression or activity of MIS or a MIS receptor that is selected by the screening methods described above.

The kit generally comprises a container and instructions for use. Suitable containers include, for example, bottles, bags (such as intravenous fluid bags) vials, syringes, and test tubes. The container holds a composition which is effective for diagnosing or treating such diseases or conditions. The active agent in the composition is usually a compound, polypeptide or an antibody described above for use in accordance with the invention. An instruction or label on, or associated with, the container indicates that the composition is used for diagnosing or treating such diseases or conditions. Other components including needles, diluents and buffers. Usefully, the kit may include a second container comprising a pharmaceutically-acceptable buffer, such as phosphate-buffered saline, Ringer's solution and dextrose solution.

Various aspects of the invention will now be illustrated in non-limiting ways by reference to the following examples.

### EXAMPLES

### Example 1

### Effect of MIS on Motor Neuron Survival

The inventors have previously discovered that mRNA transcripts of MIS, the type I receptors ALK3 and the MIS type II receptor MISRII are abundant in motor neurons, in addition to lesser amounts of alternative type I receptors ALK2 and ALK6 (Wang P Y et al. (2005) Proc. Natl. Acad. Sci. USA; 102(45):16421-5). Moreover, the presence of MIS, ALK3 and MISRII protein in motor neurons was shown by immunohistochemistry. The co-localization of MIS and MIS receptors in motor neurons was hypothesized as being consistent with MIS being an autocrine regulator of motor neuron function, or a paracrine regulator of motor neuron to motor neuron interactions.

To investigate the effect of MIS on motor neuron survival, a classical in vitro survival assay was used.

### Mouse Motor Neuron Culture

The spinal cords of 14-day-old (E14) embryos were dissected and incubated for 15 min at 37° C. in Dulbecco's phosphate buffered saline (DPBS, pH=7.2, Sigma) containing 10 μM β-mercaptoethanol (Sigma), 0.05% trypsin (Sigma) and 0.04% EDTA (Sigma). The digestion was then stopped by the addition of 0.033% trypsin inhibitor (Sigma) and the cords were mechanically dissociated by drawing them up-and-down several times through a 21- and then a 23-gauge needle.

The resulting cell suspension was passed through a mesh (pore size=100 μm, Sigma), overlaid onto 10.4% Optiprep (Sigma) in DPBS and centrifuged for 20 min at 400×g at room temperature. The upper phase, containing the purified motor neuron was collected and centrifuge for 10 min at 700×g at room temperature. The purified motor neurons (2000 cells/cm²) were cultured under serum-free conditions in Neurobasal medium (Invitrogen) supplement with B27 and 500 μM glutamine at 37° C. in a humidified atmosphere with 5% CO₂.

Recombinant MIS and hGDNF (a known motor neuron survival factor, Alomone Labs, Israel) were added immediately after seeding. Twenty-four hours later, the numbers of surviving motor neurons with axons were counted in three randomly selected fields using a phase-contrast microscope.

Half the volume of medium was changed after two days. Four days after plating, the cultures were stained with antibody to the motor neuron marker, anti-Islet-1 (39.4D5, Developmental Studies Hybridoma Bank) and the immunoreactivity developed as above, using biotinylated-anti-mouse IgG (Jackson ImmunoResearch Laboratories, USA). The numbers of surviving motor neurons were determined by counting Islet-1^{+ve} neurons with a neurite in three randomly selected fields in each well. Three wells were used for each concentration of factor and experiment was replicated four times. Virtually all cells were stained by anti-Islet antibody indicating the cultures were pure.

Purification of rhMIS

Bioreactive rhMIS was immunoaffinity-purified from CHO cells as described earlier (Ragin et al., Protein Expression Purif., 3:236-45, 1992) and its potency validated in a MIS-specific Müllerian duct regression assay (MacLaughlin et al., Methods Enzymol. 198, 358-69, 1991). The MIS produced by this method is 140 kDa (70 kDa disulfide linked homodimer) that has been activated by proteolytic processing prior to secretion. The 25-kDa carboxyl-terminal fragment, in which bioactivity resides, remains in non-covalent association with the amino terminus.

### In Vitro Experimental Results

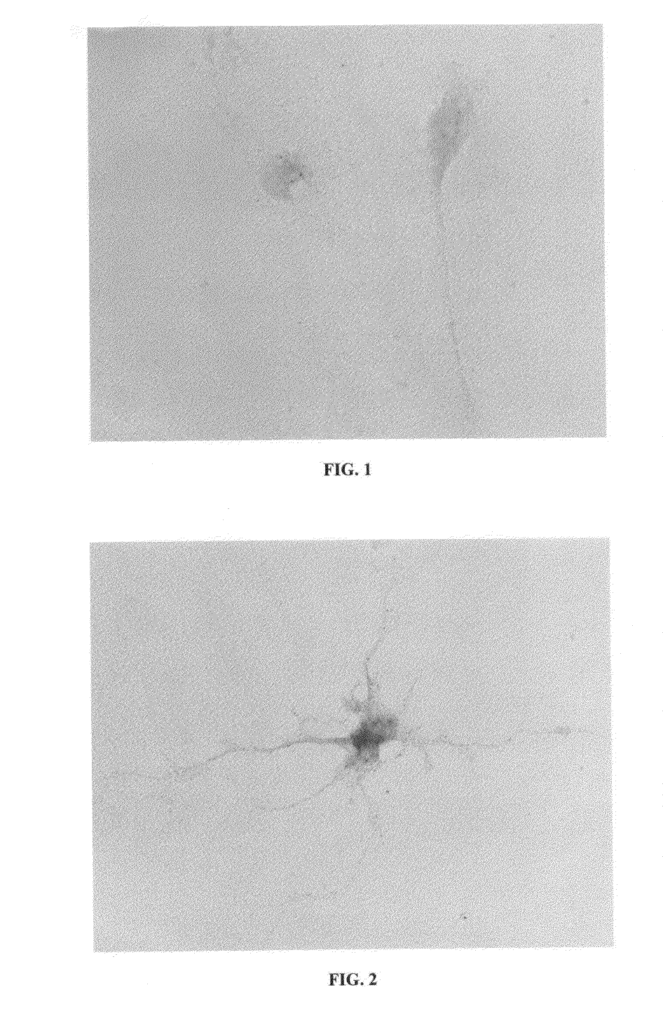

In cultures with no added growth factor, approximately half of the neurons had died and floated into the medium, by 24 hours. Most of the remaining neurons lacked neurites, and may have also been dead. A few of these neurons had extended a simple neurite, that lacked branches (FIG. 1).

After MIS treatment, there was a dose dependent increase in the number of neurons. Most of the neurons had multiple neurites, which were usually highly branched (FIG. 2). Neurons with this morphology were not seen in control cultures.

The effect of MIS was biphasic, with the maximum effect being at a concentration of 50-100 ng/ml. The MIS-induced survival persisted for up to 2 weeks, the longest time tested (not illustrated).

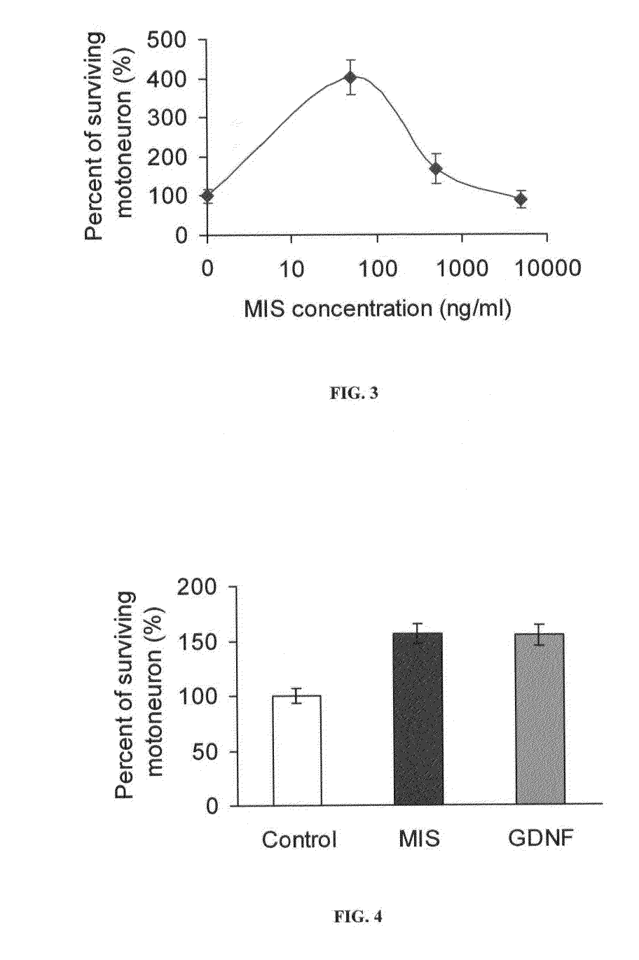

The number of neurons with neurites is illustrated in FIG. 3. The inhibitory effect of very high non-physiological doses of MIS occurs with other members of the TGF-β superfamily. The maximal effect of MIS was greater than that produced by 10 ng/ml of GDNF.

Historic assessments of putative neuronal survival factors have variably counted differentiated neurons and total neuron number. The latter method gives lower signal to noise ratios, as it includes healthy and dying neurons. Nevertheless the conclusions reached with the two methods are usually comparable. Total cell counts are illustrated from a replicate experiment in FIG. 4. The total number of surviving neurons was similar in the cultures treated with 50 ng/ml of MIS or 50 ng/ml of GDNF.

This mouse motor neuron culture experiment demonstrates that MIS is a survival factor for motor neurons in vitro. The inventors have now surprisingly identified MISRII receptors in a number of other neurons, including those of relevance to neurodegenerative diseases such as Alzheimer's disease, Parkinson's disease, Huntington's disease, Friedreich's ataxia, cerebellar ataxia, other brain disorders such as bipolar disorder, epilepsy, schizophrenia, depression, mania, autism, ADHD, brain trauma injuries and stroke. Consequently, it was hypothesized MIS is a potential survival factor for any neuron which expresses MISRII, and to have utility in treating conditions where neurons are dysfunctional and/or degenerating.

The aim of Examples 2 and 3 was to determine which neurons express type II Müllerian Inhibiting Substance receptors (MISRII).

### Example 2

### Localisation of MISRII in the Brain

### Methodology

Two approaches were used to determine the localisation of MISRII in the brain; the first involving the immunohistochemical localisation of MISRII protein and the second using Cre/LoxP fate mapping to confirm which neurons are expressing MISRII.

### Immunohistochemistry

The anti-MISRII antibody used was purchased from R&D System. The inventors have previously used this antibody to detect MISRII in spinal motor neurons (Wang P Y et al. (2005) Proc. Natl. Acad. Sci. USA; 102(45):16421-5). The presence of MISRII in spinal motor neurons has now been confirmed by multiple other techniques and by the use of a second non-commercial antibody to MISRII. These observations validate the use of the R&D antibody to detect MISRII in neurons.

Transverse sections of lumbar spinal cords and selected tissues were cut in a cryostat at a thickness of 10 μm. The sections were stained by immunohistochemistry as previously described (Russell et al., Neurosci., 97(3):575-80, 2000). Briefly, the sections were fixed in either 1% or 4% neutral buffered paraformaldehyde at 4° C. and incubated with goat anti-MISRII (1:50, R&D System), specific primary antibody overnight at 4° C.

The slides were then sequentially incubated with a biotinylated anti-goat IgG antibody (1:75, Sigma, St Louis, Mo., USA), methanol/H₂O₂, and finally a streptavidin biotinylated-horseradish peroxidase complex (1:200, Amersham). The immunoreactivity was visualized using 3-amino-9-ethylcarbamide as the chromogen (Sigma). In each study, the primary antibody was replaced by a non-immune goat IgG (Sigma) as a control for non-specific binding. Standard washes between each step were used (Russell et al., Neurosci., 97(3):575-80, 2000).

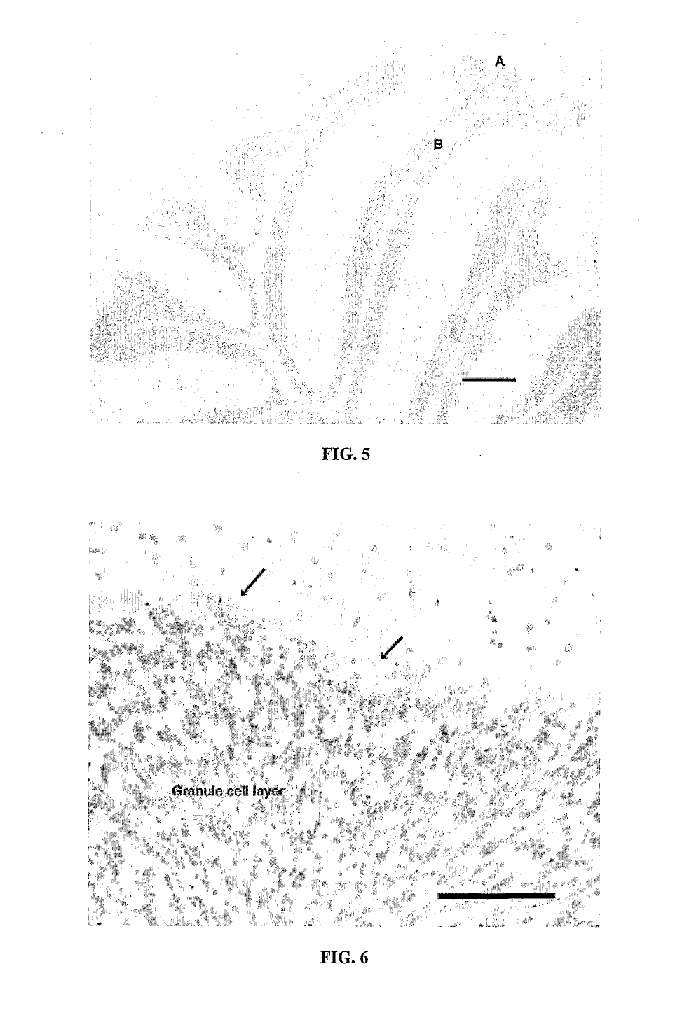

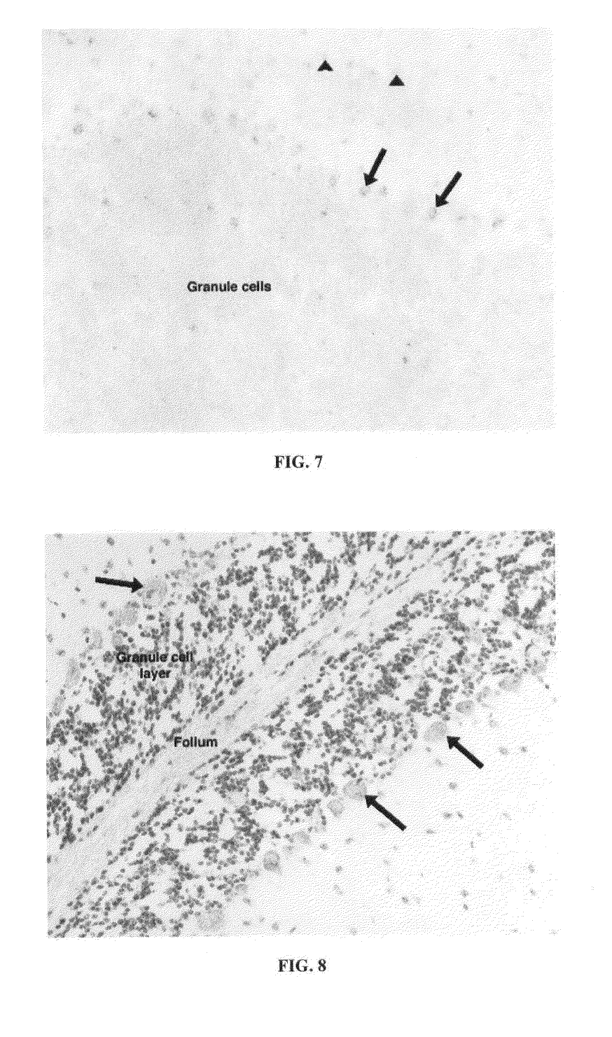

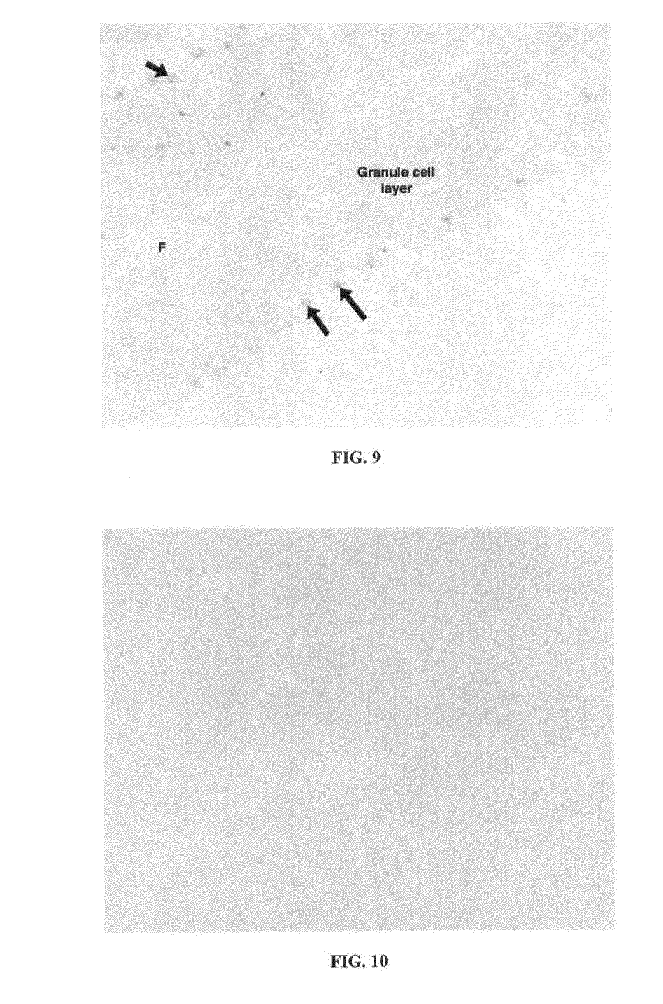

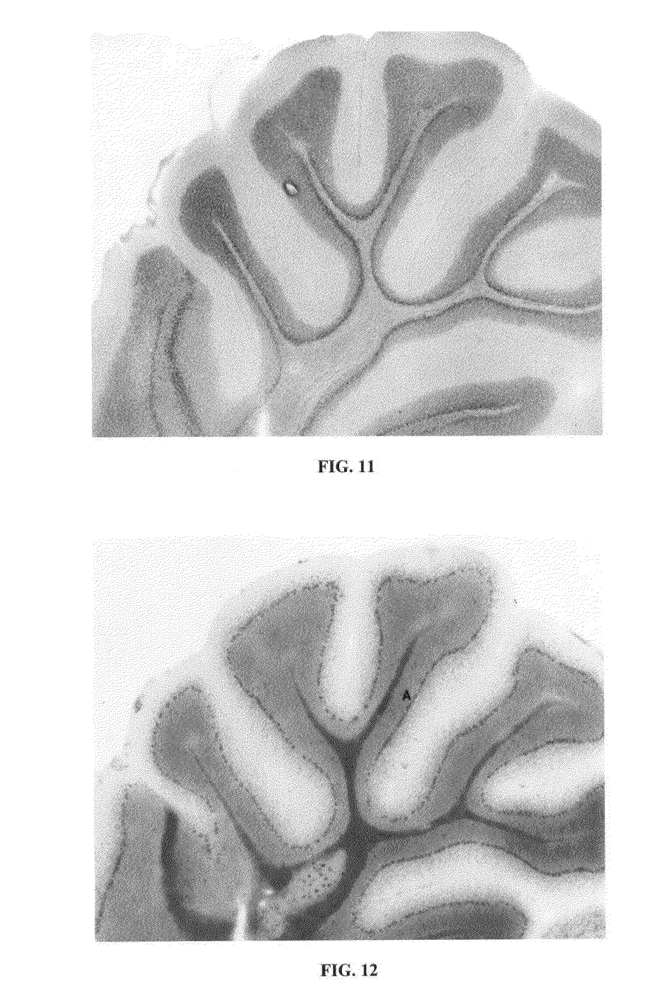

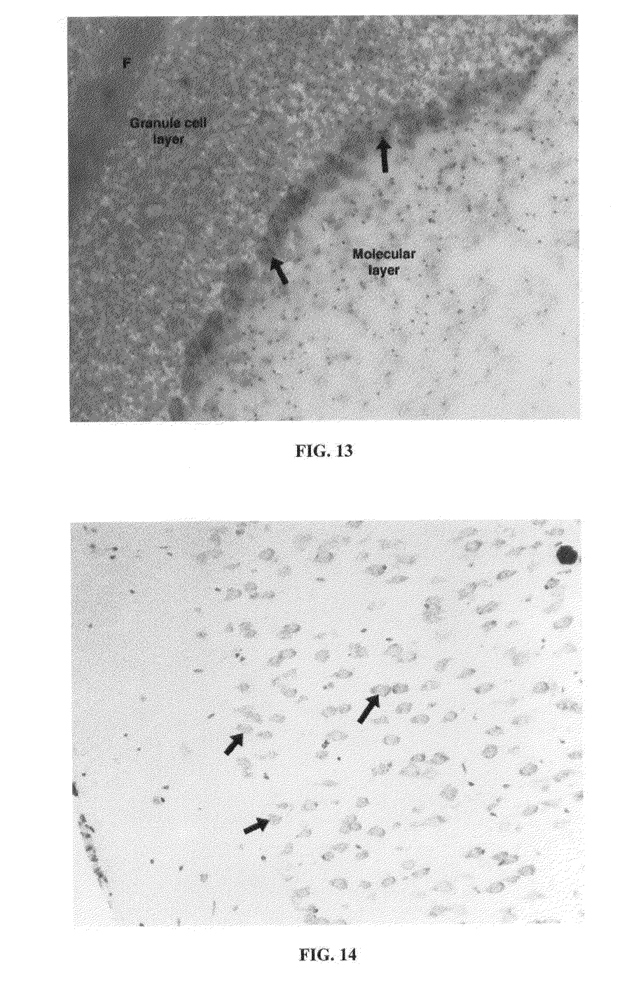

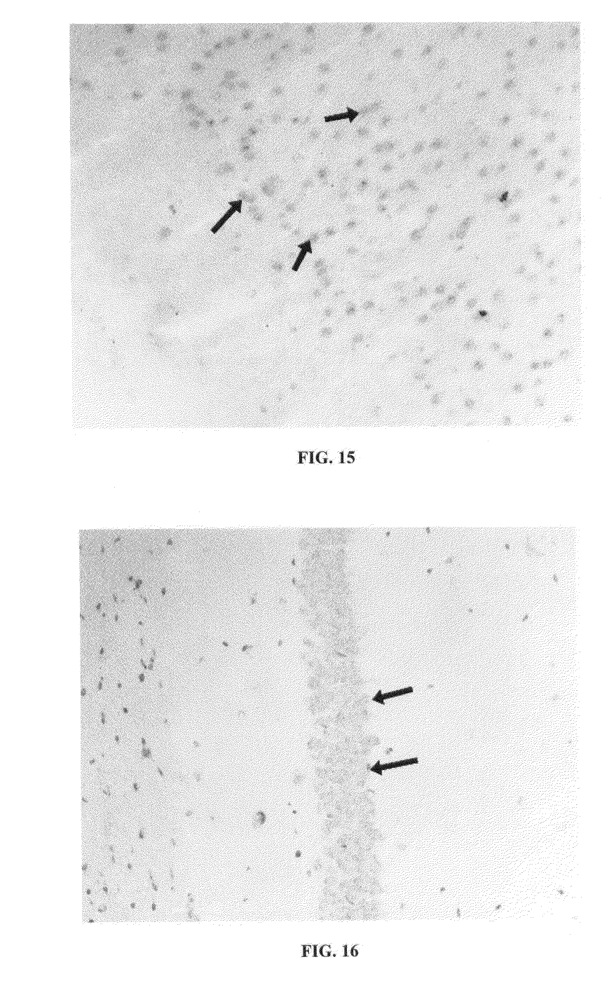

Sections of cerebellum, cerebral cortex and hippocampus of murine brain stained with either cresyl violet to show neurons, an antibody to MISRII or a non-immune IgG are shown in FIG. 5, FIG. 6, FIG. 7, FIG. 8, FIG. 9, FIG. 10, FIG. 11, FIG. 14, FIG. 15, FIG. 16 and FIG. 17 as described below.

### Cre/LoxP Fate Mapping

Genetically-modified mice were used to confirm the MISRII-immunohistochemistry results using Cre/Lox fate mapping.

The basic strategy for Cre/loxP-directed gene knockout experiments is to flank (‘flox’) a gene or sequence of interest with two 34 base pair loxP sites followed by the use of the site-specific Cre-recombinase to excise the intervening DNA between the LoxP sites, thus generating a null allele in all cells where Cre is expressed.

Expression of Cre-recombinase can be achieved by crossing mice carrying the ‘floxed’ target gene with transgenic Cre-expressing mice.

The mice used in this example were originally generated by Prof. R. Behringer (MISRII-Cre) and by Prof. Herbison (ROSA26-LacZ; Soriano, Nat. Genet., 21:70-71, 1999).

The MISRII-Cre mice have one normal copy of the MISRII gene. In the other the coding region of MISRII has been replaced by a Cre-recombinase gene. Consequently, these mice express Cre-recombinase whenever and wherever MISRII is expressed.

The reporter mouse line is ROSA26-LacZ. The ROSA26-LacZ mouse line contains the combination of a termination codon surrounded by LoxP elements and E. coli β-galactosidase gene (LacZ) under a ubiquitously active promoter. The stop codon prevents the transcription of LacZ. However, when Cre-recombinase is present, the stop codon is deleted and the cell begins to produce LacZ. LacZ can then be visualized in sections of tissues via a simple histological technique.

When ROSA26-LacZ mice are crossed with MISRII-Cre mice, 25% of the resulting RMSR pups have both genetic modifications. The cells of these RMSR pups will produce Cre-recombinase and lacZ, whenever they produce MISRII and Cre-recombinase. Once a cell produces MISRII and Cre-recombinase, the induction of lacZ is permanent as it results from a modification of the DNA. This method thus creates a permanent record of cells that have produced, or are producing, MISRII.

Sections of cerebellum from a RMSR mouse stained with β-galactosidase to reveal the location of LacZ expression are shown in FIG. 12, FIG. 14 and FIG. 18, as described above.

### Results

The results obtained by the two methods are in good agreement. The type of neurons which are stained by the antibody using immunohistochemistry also produce lacZ in the RMSR mice. These results unambiguously show a number of types of neurons produce MISRII.

Neurons showing MISRII-positive expression include:
Spinal cord (highest level of expression)
lateral and medial motor neurons (strong)
other spinal neurons (moderate to weak)
Cerebellum (mostly weak)
Purkinje cells (moderate)
Brainstem (next highest level after spinal cord)
Brainstem motor neurons (strong to moderate)
Dorsal nucleus of vagus (moderate)
Spinal trigeminal nuclei (moderate)
Lateral reticular nuclei (strong)
Inferior olive (strong)
Paramedian reticular nucleus (moderate)
Lateral paragigantocellular nuclei (moderate)
Cochlear and vestibular nuclei (moderate)
Vestibulocochlear nuclei (moderate)
Gigantocellular reticular nucleus (moderate)
Paraolivary nuclei (moderate)
Nucleus of trapezoid body (moderate)
Midbrain (lower region of expression)
Substantia nigra (moderate).
Various other neurons, identity to be confirmed (weak)
Cerebral cortex (moderate to low level of expression)
Hippocampus, including pyramidal cells of CA and granular cells of dentate gyrus (brighter than other cortical neurons, but only moderate/weak compared to motor neurons).
Weak to moderate staining of various neurons, to be identified.
Thalamus/Hypothalamus (weak)

The choroid plexus was stained in the RMSR mice (FIG. 18). The choroid plexus of wild-type mice was also stained by the anti-MISRII antibody. The intensity of the stain in the choroid plexus by immunohistochemistry was greater than that of any neuron.

### Example 3

### MISRII mRNA in the Brain

The brains of adult mice were dissected into their major parts, and the mRNA within them extracted and analyzed by PCR to determine the levels of MISRII mRNA expression.

RNA Preparation, cDNA Synthesis and Real-Time Polymerase Chain Reaction (PCR)

Total RNA fractions were isolated using TRIzol reagent (Invitrogen, Carlsbad, Calif., USA) for adult mouse tissues or PicoPure™ RNA isolated kit (Arcturus Engineering) for LCM samples following the manufacturer's protocols. The isolated RNA fractions were initially treated with DNase I (Promega, Madison, Wis., USA) to remove genomic DNA contamination. The cDNA was synthesized with SuperScript II RNaseH⁻ (Invitrogen) and oligo-d(T)₁₅ as the primer. The real-time PCR reactions were performed using an ABI Prism 7000 (Applied Biosystems), SYBR Green Master Mix (Applied Biosystems) and gene specific primers (Table 1).

TABLE 1
Oligonucleotide primer sequences

| | | | |
|---|---|---|---|
| Primer | Sequence | Accession # | Size (bp) |

| | | | |
|---|---|---|---|
| MISRII-F | 5′-CAGAGTTCTTGTCTCCTCAGTGTT-3′ | NM_144547 | 158 |
| MISRII-R | 5′-AGAGAATGACAGCAGTAGGCATGT-3′ | | |
| GAPDH-F | 5′-CTTCATTGACCTCAACTA-3′ | NM_008084 | 300 |
| GAPDH-R | 5′-TTCACACCCATCACAAAC-3′ | | |

The forward and reverse primers used for RT-PCR detection of the listed genes are indicated by “-F” and “-R”, respectively.

A two-step PCR reaction was carried out with denaturation at 95° C. for 15 seconds, annealing and extension combined at 60° C. for one minute in a total of 40-50 cycles. The uniqueness of amplicons was analyzed using dissociation curves and by sequencing (Centre for Gene Research, University of Otago). The copy numbers of each gene was calculated from a standard curve. The DNA used for each standard was amplified from a mouse spinal cord cDNA pool using PCR reactions and purified using gel electrophoresis and QIAquick Gel Extraction Kit (Qiagen, Valencia, Calif., USA) following the manufacturer's protocol.

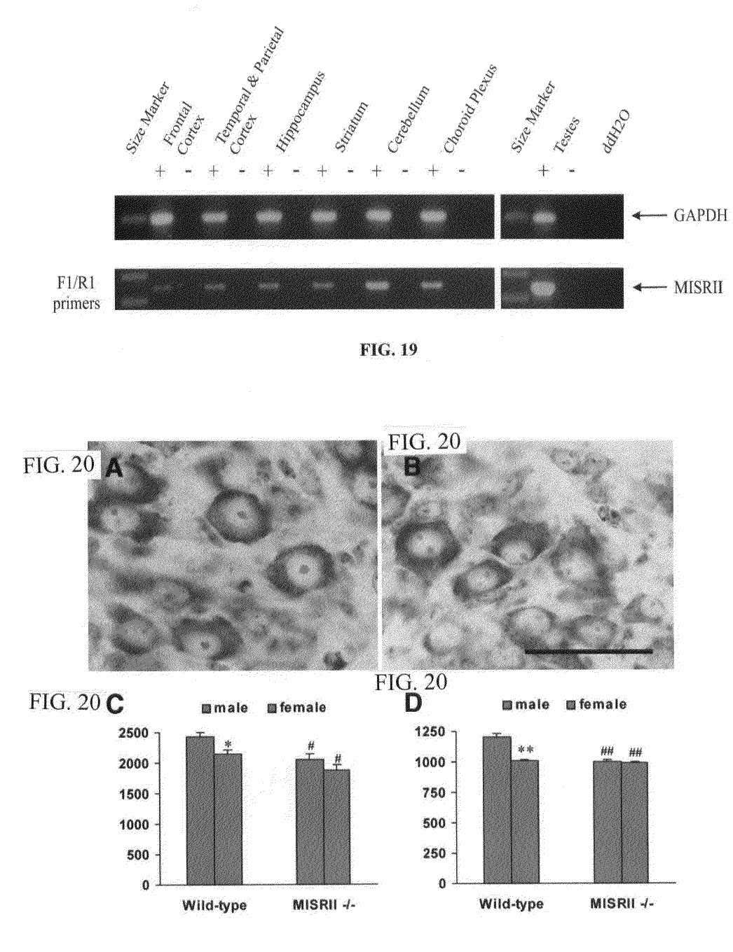

As shown in FIG. 19, all parts of the brain contained the mRNA for MISRII, although the various regions of the brain had differing amounts of MISRII. The cerebellum was a particularly rich source, whereas the frontal cortex had lesser amounts. This data is consistent with that obtained from Example 2 in which the MISRII-lacZ mice showed expression of MISRII in all neurons. It is also consistent with the immunohistochemistry data of Example 2 which showed that all neurons contain MISRII protein, but that each type of neuron had a different abundance.

### Discussion

The inventors have identified MISRII expression, which is the unique receptor for MIS, in a number of types of neurons. These findings strongly suggest that MIS is involved in the regulation of these neurons. Furthermore, MIS supported the survival and differentiation of embryonic motor neurons in vitro, indicating that activation of MIS receptors leads to downstream functional consequences in neurons expressing MISRII.

In the TGF-β superfamily, the type II receptor determines ligand and specificity, whereas the type I receptors controls downstream pathway activation. Bone morphogenic proteins (BNPs) also activate the ALK3 type I receptor in combination with BMPRII, and are known to be potent neuronal survival factors.

Without wishing to be bound by theory it is considered that MIS also affect neurons by activation of the type II Müllerian inhibitory substance receptor (MISRII) and a type I co-receptor. The presence of MIS receptors in these neurons indicates MIS may be important in various neurodegenerative conditions and/or in psychiatric disorders.

The motor neurons of the spinal cord and brainstem produce the highest levels of MISRII, as evidenced by immunohistochemistry. Various other type of neurons are stained with moderate intensity, with an even wider range of neurons having low but clear levels of MISRII expression.

The low expression of MISRII by some neurons may simply reflect their small size. Consequently, the broad distribution of MISRII on neurons indicates that MIS or a MIS analog is useful in treating injuries that affect a region of the brain. Such injuries would include external trauma or damage resulting from stroke.

Of particular interest was that MISRII expression was detected in neurons of the substantia nigra (of relevance to Parkinson's disease), cerebral cortex and hippocampus (of relevance to Alzheimer's disease) indicating MIS is useful in developing new treatments for these diseases.

Mood disorders involve changes to the cerebral cortex, cerebellum, caudate and putamen (Soares and Mann, Biol. Psych., 41:86-106, 1997). The production of MISRII by neurons in these structures indicates that these neurons are responsive to MIS, and may indicate that MIS can slow or reverse the changes occurring in mood disorders.

The highest level of MISRII expression was found in the choroid plexus, which is involved in the secretion and transport of molecules from the blood to the cerebrospinal fluid. The MIS in blood has generally been considered to be leakage/waste from the gonads. The current finding further supports the role of MIS in the brain and offers an alternative route for MIS to be transported from the blood into the cerebrospinal fluid.

The ability of gonadal MIS to enter the cerebrospinal fluid suggests MIS may also be used to treat patients who have menopause, who have suffered unilateral or bilateral loss of a gonad and/or whose gonads have age- or disease-related dysfunction. There is clear evidence of a gonad-brain connection. Women, for instance, who have had both their ovaries removed are twice as likely to develop Parkinson's disease.

MIS can be provided to menopausal women, in order to protect them against degradation of the functions of the brain. It may be necessary to do this in conjunction with hormonal-replacement therapy. Similarly, the testes and ovaries are sometimes removed for various medical reasons (e.g., cancer). MIS-therapy may also be used to prevent such patients against neural degradation, along with an androgen, estrogen and/or progesterone.

Given that MIS is a male specific hormone in embryos, and that motor neurons express its receptors during development it was hypothesized that the motor neurons in the spinal cord should be sexual dimorphic, with a male bias, and that this sexual dimorphism should be absent in mice that lack MISRII or MIS and are described in Examples 4 and 5, respectively.

### Example 4

### Loss of Motor Neurons in MISRII^{-/-} Mice

### Animals

The University of Otago's Animal Ethics Committee approved all experiments. C57Bl6 mice were bred and maintained in M.I.C.E.™ cages (Animal Care Systems, Littleton, Colo.) and their food sterilized by gamma irradiation. The room had a 14 h white light/10 h dark-sodium light phase [McLennan I S & Taylor-Jeffs J (2004) Laboratory Animals 38:1-9] with the dark phase beginning at 1 pm.

### Methods

Mice with a null mutation (MISRII^{-/-}) [Jamin S P et al., (2002) Nature genetics 32:408-410] were obtained by breeding MISRII^{+/-} studs and dams. The resulting pups were killed at birth, and fixed with formalin as described below.

The genotype of the mice was determined by polymerase chain reaction (PCR). The primers used for genotyping are: MISRII-F1 5′-CTTCCCACATAGCTCCCT TGTCT-3′ and MISRII-R1 5′-GAACCTCCAGGAGTGCCACAG-3′ to generate a 230 base pair PCR product for the wild type alleles; Cre-F1 5′-GTTGATGCCGGTGAACGTGCAAA-3′ and Cre-R1 5′-ATCAGCTACACCAGAGACGGAAA-3′ to generate a 320 bp null mutant alleles. The gender of neonatal mice was determined by a male specific gene, the sex-determining region of chromosome Y (SRY) using primers: Sry-F2 5′-TCTTA AACTC TGAAG AAGAG AC-3′ and Sry-R2 5′-GTCTT GCCTG TATGT GATGG-3′ (UniSTS #144593, sourced from http://www.ncbi.nlm.nih.gov/genome/sts/sts.cgi?uid=144593) to generate a 380 base pair PCR product.

The number and size of the lumbar lateral motor column motor neurons was determined using rigorous stereological techniques [Gundersen H J et al., (1988) Acta Pathologica Microbiologica et Immunologica Scandinavica 96:857-881]. The person undertaking the cell counts was blind to the sex and genotype of the mouse.

Newborn pups were skinned and fixed in 10% neutral buffered formalin. The spinal cords were dissected and embedded in Technovit (Kulzer & Co). Sections were stained overnight with 0.001% cresyl violet acetate. Motor neurons in the lateral motor column of the lumbar spinal cord were counted blind and used the optical dissector method as described previously [Day W A et al., (2005) Neurobiol Dis 19:323-330].
Every fifth section were counted and the number were multiplied by five to give an estimated total cell counts. The diameters of motor neurons were measured in at least 100 cells from 6 or 7 animals in the middle level of the lumbar spinal cord. One nuclear diameter was measured along an axis passing through a nucleolus and another diameter along a secondary axis at a right angle to the center of the nucleolus. The two diameter values were then averaged and the nuclear volume calculated using the formula: Volume= 4/3πr³.

### Results

In wild-type neonates, the number of motor neurons in the lumbar lateral motor column of male mice was 15% (P=0.01) greater than in female mice (FIG. 20C). The nuclei of male motor neurons were approximately 20% (P<0.001) larger than their female counterparts (FIG. 20D).

The morphology of motor neurons in the lumbar lateral motor column of male wild type (FIG. 20A) and male MISRII^{-/-} (FIG. 20B) neonates. Male mice of MISRII^{-/-} have 18% (P=0.01) fewer motor neurons than wild-type males and the mean size of their motor neurons was 20% smaller (P<0.001) (FIG. 20C and FIG. 20D). The number and size of motor neurons in the MISRII^{-/-} males was not different to those of MISRII^{+/+} females.

### Conclusions

The lumbar lateral motor column is dimorphic as predicted. The observation that the dimorphism is absent in mice with defects in MIS signaling is compelling evidence for MIS being a regulator of motor neuron survival in living animals.

The testes are the only known source of MIS in an embryo. Wang P Y et al. (2005) have shown that MIS is produced by adult neurons, but that embryonic neurons do not stain with anti-MIS antibodies. This indicates that embryonic neurons produce little or no MIS. Consequently, the observation that the number of motor neurons is altered in MISRII^{-/-} mice is strong evidence that MIS from the testes affects the brain. The known presence of MIS in male fetal and neonatal blood adds considerable weight to this conclusion.

### Example 5

### Loss of Motor Neurons in MIS^{-/-} Mice

In Example 4 it was shown that neonatal male mice have more motor neurons in their lumbar lateral motor column than their female littermates. The sizes of the motor neurons were also larger in the males. This dimorphism is absent in mice that have a null mutation in the type II MIS receptor MISRII, and are therefore unresponsive to MIS.

The characteristics of mice with a null mutation of their MIS gene (MIS^{-/-}) were examined as a further test of the importance of MIS.

### Methods

Mice with a null mutation in the MIS gene as described in Behringer et al., Cell 79:415-425 (1994) were obtained from Jackson Laboratories (USA). The mice were bred and processed as described in Example 4.

### Results

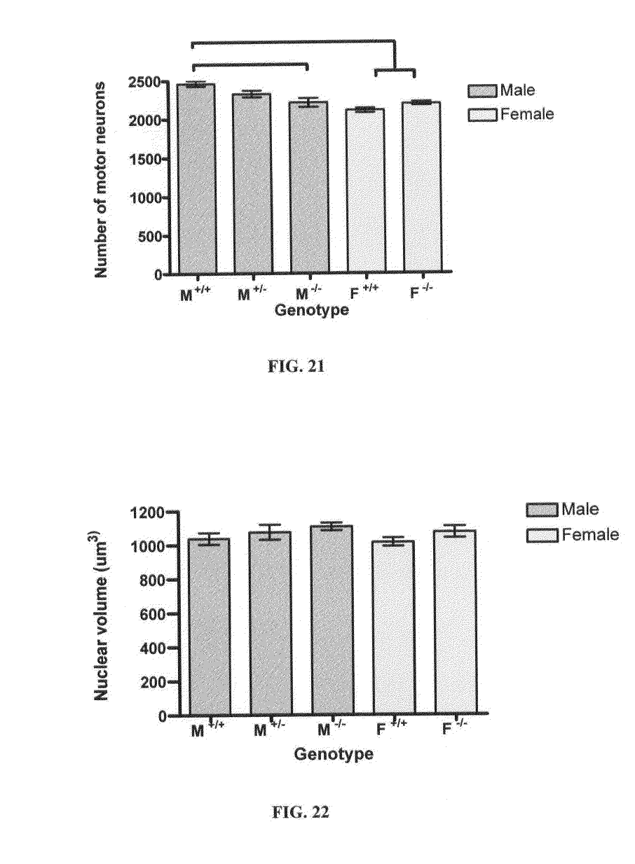

The number of motor neurons in the lumbar spinal cord of wild type from the MIS^{+/-} colony was dimorphic, with a male bias of approximately 10% (FIG. 21). This is similar to the bias observed in the MISRII^{+/-} colony of Example 4.

The dimorphism was absent in the mice lacking MIS (MIS^{-/-}). That is, the number of motor neurons in MIS^{-/-} males was not statistically different to that in females, and was significantly different to wild type (MIS+/+) males. In contrast to the MISRII^{+/-} colony of Example 4, the size of the motor neurons was not dimorphic (FIG. 22).

### Conclusions

The absence of dimorphism in the MIS^{-/-} mice replicates the observations seen in the MISRII^{-/-} mice in Example 4, providing compelling proof that MIS is a motor neuron survival factor in vivo.

Many neurons survive in the MIS^{-/-} and MISRII^{-/-} mice. This is to be expected as the survival of motor neurons is controlled by multiple factors [Oppenheim R W (1996) Neuron 17:195-197] which come from the various cell types that motor neuron interact with, including muscle fibres and Schwann cells. The function of MIS in the blood during development is to create a male bias. Critically, 100% of the male bias is lost in the MIS^{-/-} and MISRII^{-/-} mice.

In the adult, MIS ceases to be dimorphic (Lee et al., supra; Wang et al., supra). Hence, its effects will cease to be male specific and should be important to both adult males and females.

The size of the motor neurons was not dimorphic in the MIS^{+/-} colony. The degree of dimorphism varies between different mouse colonies, for reasons that are unknown. The critical circumstances could include genetic background and physiological or pathological context.

Those persons skilled in the art will understand that the above description is provided by way of illustration only and that the invention is not limited thereto.

### INDUSTRIAL APPLICATION

The methods, uses and pharmaceutical compositions of the present invention have utility in the modulating neuron function and activity, through the administration of at least one MIS receptor agonist or antagonist. Such embodiments are applicable particularly but by no means exclusively to treat neurodegenerative diseases and other brain disorders.

Those persons skilled in the art will understand that the above description is provided by way of illustration only and that the invention is not limited thereto.

1. A method of
(a) treating a condition or disease characterized by neuronal cell death or neural cell impairment in a patient in need thereof;
(b) modulating neuronal cell function in a patient in need thereof; or
(c) enhancing neuronal cell survival in a patient in need thereof,
said method comprising, administering to said patient an effective amount of at least one Müllerian inhibitory substance (MIS) receptor (MISR) agonist or antagonist.

2-101. (canceled)

102. The method of claim 1, wherein enhancing said neuronal cell survival in a patient in need thereof comprises providing to a population of neural cells of said patient an amount of at least one Müllerian inhibitory substance (MIS) receptor (MISR) agonist or antagonist effective to enhance said neuronal cell survival in said patient.

103. The method of claim 1, wherein said condition or said disease is selected from the group consisting of Alzheimer's disease, Parkinson's disease, Huntington's disease, Friedreich's ataxia, Kennedy's disease, cerebellar ataxia, bipolar disorder, epilepsy, schizophrenia, depression, mania, autism, ADHD, multiple sclerosis, brain trauma injuries and stroke.

104. The method of claim 102, wherein said neural cells are comprised within the cerebellum, the midbrain or the forebrain.

105. The method of claim 1, wherein said MISR is a MIS type II receptor (MISRII), a MIS type I receptor (MISRI), or a combination of both MISRII and MISRI.

106. The method of claim 105, wherein said MISR is a MISRII.

107. The method of claim 1, wherein said MISR agonist is selected from the group consisting of: (a) MIS; (b) an antibody or an antibody fragment that binds to MIS; or (c) a MISR or a MISR subunit.

108. The method of claim 1, wherein said MISR antagonist is an inactive variant of MIS; MISR; an antisense sequence; an siRNA; a ribozyme; or an antibody or antibody fragment that binds to MIS, a MISR or a MISR subunit.

109. The method of claim 1, wherein said MISR agonist or antagonist is formulated for delivery to a mammalian brain.

110. The method of claim 1, wherein said MISR agonist or antagonist is formulated for systemic administration.

111. The method of claim 1, wherein said patient has menopause, suffers from an age- or disease related-dysfunction of the gonads, or has suffered unilateral or bilateral loss of a gonad.

112. The method of claim 1, wherein said patient is human.

113. The method of claim 1, wherein said MISR agonist or antagonist comprises an at least 20 amino acid contiguous sequence from a polypeptide comprising a sequence selected from the group consisting of SEQ ID NO:2, SEQ ID NO:4, SEQ ID NO:6 and SEQ ID NO:8.

114. The method of claim 113, wherein said MISR agonist or antagonist comprises the amino acid sequence of SEQ ID NO:2, SEQ ID NO:4, SEQ ID NO:6 or SEQ ID NO:8.

115. The method of claim 1, wherein said MISR agonist or antagonist comprises a peptide or polypeptide sequence that is encoded by an at least 60 base pair contiguous nucleotide sequence from a polynucleotide comprising a sequence selected from the group consisting of SEQ ID NO:1, SEQ ID NO:3, SEQ ID NO:5 and SEQ ID NO:7.

116. The method of claim 115, wherein said MISR agonist or antagonist comprises a peptide or polypeptide sequence that is encoded by a nucleotide sequence that comprises the sequence of SEQ ID NO:1, SEQ ID NO:3, SEQ ID NO:5 or SEQ ID NO:7.

117. The method of claim 1, wherein said MISR agonist or antagonist is administered in conjunction with at least one additional active compound that is selected from the group consisting of a neurotrophic factor, a glial cell line-derived neurotrophic factor (GDNF), a brain-derived neurotrophic factor (BDNF), a ciliary-derived neurotrophic factor (CNTF), glutamate, a gonadal hormone, estrogen, progesterone, androgen and synthetic equivalents thereof.

118. A composition comprising: (a) at least one MISR agonist or antagonist that modulates neuronal cell function or enhances neuronal cell survival in a patient in need thereof; and (b) a pharmaceutically-acceptable carrier or excipient.

119. The composition of claim 118, wherein said neurons are comprised within Purkinje cells, the substantia nigra, the cerebral cortex, the caudate, the putamen, the hippocampus, the hypothalamus or the thalamus.

120. The composition of claim 118, wherein said MISR is a MISRII, a MISRI, or a combination of both MISRII and MISRI.

121. The composition of claim 120, wherein said MISR is a MISRII.

122. The composition of claim 118, wherein said MISR agonist is MIS, or a functional derivative thereof; an antibody or an antibody fragment that binds to MIS or a functional derivative thereof; or a MISR or MISR subunit.

123. The composition of claim 118, wherein said MISR antagonist is an inactive variant of MIS; MISR; an antisense sequence; a siRNA; a ribozyme; or an antibody or antibody fragment that binds to MIS, a functional derivative thereof, or a MISR or MISR subunit.

124. The composition of claim 118, wherein said MISR agonist or antagonist is formulated for delivery to a mammalian brain.

125. The composition of claim 118, wherein said MISR agonist or antagonist is formulated for systemic administration.

126. The composition of claim 118, wherein said patient is human.

127. The composition of claim 118, wherein said MISR agonist or antagonist comprises an at least 40 amino acid contiguous sequence from any one of SEQ ID NO:2, SEQ ID NO:4, SEQ ID NO:6 or SEQ ID NO:8.

128. The composition of claim 118, wherein said MISR agonist or antagonist comprises the amino acid sequence of any one of SEQ ID NO:2, SEQ ID NO:4, SEQ ID NO:6 or SEQ ID NO:8.

129. The composition of claim 118, wherein said MISR agonist or antagonist comprises a peptide or polypeptide sequence that is encoded by an at least 120 base pair contiguous nucleotide sequence selected from any one of SEQ ID NO:1, SEQ ID NO:3, SEQ ID NO:5 or SEQ ID NO:7.

130. The composition of claim 129, wherein said MISR agonist or antagonist comprises a peptide or polypeptide sequence that is encoded by a nucleotide sequence selected from the group consisting of SEQ ID NO:1, SEQ ID NO:3, SEQ ID NO:5 and SEQ ID NO:7.

131. The composition of claim 118, formulated for simultaneous, separate or sequential administration with at least one additional active compound selected from the group consisting of neurotrophic factors including but not limited to glial cell line-derived neurotrophic factor (GDNF), brain derived neurotrophic factor (BDNF), ciliary derived neurotrophic factor (CNTF), glutamate, and gonadal hormones including but not limited to estrogen, progesterone, androgen and synthetic equivalents thereof.

132. A method of diagnosing a condition or disease characterized by neuronal cell death or impairment or a predisposition to developing said condition or disease in a patient, said method comprising at least the step of determining the level of MIS or MISR in a patient sample, wherein an alteration in the level of MIS compared to a control level indicates that the patient has, or is at risk of developing, a condition or disease characterized by neuronal cell death or impairment.

133. The method of claim 132, wherein the level of MIS or MISR is the expression level of MIS or MISR.

134. the method of claim 132, wherein said condition or said disease is selected from the group consisting of Alzheimer's disease, Parkinson's disease, Huntington's disease, Friedreich's ataxia, Kennedy's disease, cerebellar ataxia, a brain disorder, bipolar disorder, epilepsy, schizophrenia, depression, mania, multiple sclerosis, autism and ADHD.

135. The method of claim 132, wherein said patient has menopause, suffers from an age- or disease related-dysfunction of the gonads, or has suffered unilateral or bilateral loss of a gonad.

136. The method of claim 132, wherein said patient sample is selected from the group consisting of tissue, blood, lymph, cerebrospinal fluid, urine and ejaculate.

137. A method of screening for a compound that modulates neuronal cell function or survival, said method comprising at least the steps of:
(a) contacting a test cell that expresses MIS or MISR with a test compound;
(b) determining the expression level of MIS or MISR; and
(c) selecting the compound that modulates the expression level compared to that in the absence of the test compound.

138. A method of screening for a compound that modulates neuronal cell function or survival, said method comprising at least the steps of:
(a) contacting a test compound with a MIS or MISR polypeptide;
(b) detecting the biological activity of said MIS or MISR polypeptide; and
(c) either: (i) selecting the compound that modulates the biological activity of the polypeptide in comparison with the biological activity detected in the absence of the compound; or (ii) selecting the compound that binds to the polypeptide.

139. A compound that alters expression or activity of MIS or MISR identified by a screening method in accordance with claim 137 or claim 138.
